(19) United States
(12) Patent Application Publication
(10) Pub. No.: US 2024/0197341 A1
(43) Pub. Date: Jun. 20, 2024

(54) Method and Device for Endoscopic Aspiration

(71) Applicant: EndoTheia, Inc., Nashville, TN (US)

(72) Inventors: Robert James Webster, III, Nashville, TN (US); Joshua Ball Gafford, Nashville, TN (US); Scott Joseph Webster, Nashville, TN (US); Patrick Louis Anderson, Nashville, TN (US); Stanley Duke Herrell, III, Nashville, TN (US); Daniel Caleb Rucker, Nashville, TN (US)

(21) Appl. No.: 18/082,435

(22) Filed: Dec. 15, 2022

(51) Int. Cl. A61B 17/22; A61B 1/00; A61B 1/005; A61B 1/015; A61B 1/018; A61B 1/05; A61B 1/06; A61B 1/307

(52) U.S. Cl. CPC A61B 17/22; A61B 1/00135; A61B 1/0052; A61B 1/0057; A61B 1/015; A61B 1/018; A61B 1/05; A61B 1/0676; A61B 1/307; A61B 2017/00561

(57) ABSTRACT

An endoscope device is provided. The device includes a handle, a steerable sheath, disposed on the handle, and a flexible tube. The device further includes a light source and an image sensor disposed on the flexible tube. The handle includes a passage. The steerable sheath includes a tip, a steerable section, and a lumen. The steerable section of the steerable sheath is actuable to form a bend, such that the tip is operable to be steered toward an anatomical region within a patient. The flexible tube is configured to be inserted within the lumen and the inner passage, such that the light source and the image source are positioned at or near the tip of the steerable sheath. The flexible tube is further configured to be removed from the lumen, such that the lumen is operable to aspirate a bodily object from the anatomical region via the tip.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### CROSS-REFERENCES TO RELATED APPLICATIONS

Not Applicable.

### STATEMENT REGARDING FEDERALLY SPONSORED RESEARCH OR DEVELOPMENT

Not Applicable.

### REFERENCE TO SEQUENCE LISTING OR COMPUTER PROGRAM LISTING APPENDIX

Not Applicable.

### BACKGROUND

The present disclosure relates generally to devices and methods for endoscopic surgery, and more particularly to providing endoscopic devices for performing minimally invasive endoscopic removal of objects from within a patient.

Each year three-and-a-half million people suffer from kidney (e.g., renal) stones (e.g., calculi). In general, and as used herein, “stones” or “calculi” are a class of bodily objects that are hard deposits made of minerals and salts that form inside the body. Kidney stones, then, are a subset of such stones that form within the kidneys (and the calyces located therein) in particular. One out of every five conditions involving kidney stones (approximately seven-hundred thousand) require an intervention. Of these seven-hundred thousand patients, sixty-three percent have small kidney stones and are well-served by the present standard of care, which often includes “flexible ureteroscopy”.

Generally speaking, ureteroscopy is a form of endoscopy, which is a procedure in which an instrument is introduced into the body (through a natural orifice or percutaneous approach) to give a view of and/or adjust its internal parts. Such instruments are typically referred to as “endoscopes,” or, where specifically configured for ureteroscopic procedures, “ureteroscopes.” Such instruments are often configured to navigate various bodily cavities, thus affording the term “flexible endoscope” or “flexible ureteroscope.” In some cases, endoscopic or ureteroscopic procedures involve removal of bodily objects including, but not limited to, stones. In particular, ureteroscopy is performed in the upper urinary tract to address (e.g., remove, in some case) kidney stones. Of course, one of skill in the art would appreciate that endoscopes are used in many other areas of the body to remove bodily objects such as stones. As examples, flexible endoscopes may also be used in the gastrointestinal (GI) tract, the abdomen, the lung(s), the brain, and so on, via natural orifices or other percutaneous approaches.

To illustrate an endoscopic procedure as applied to the particular context of ureteroscopy, the typical ureteroscopy procedure may include a ureteroscope being passed through the urethra, bladder, and then directly into the upper urinary tract in order to remove a kidney stone (or other bodily object). Depending on the size of the kidney stone to be removed, ureteroscopy procedures may require a laser lithotripsy procedure (i.e., using a laser to break kidney stones into tiny pieces) via the endoscope before “basketing” (i.e. deploying a flexible (Nitinol) ureteroscopic kidney stone basket from the endoscope to capture and remove) the pieces using the ureteroscope. In other cases, ureteroscopy procedures may simply require “dusting” (i.e. using a laser to break kidney stones into small enough pieces so as to pass naturally). As suggested above, however, such methods may be applied to the more general field of endoscopic procedures to remove stones and other bodily objects, and are not altogether particular to ureteroscopy.

As the size of kidney stones get larger, however, surgeons face a troubling dilemma in how to treat the remaining thirty-seven percent of patients (approximately two-hundred and sixty-thousand per year in the U.S.A. alone) who have larger kidney stones (e.g., eleven millimeters or larger in diameter) that make typical procedures difficult and inefficient. For example, dexterity limitations in aiming the laser for performing laser lithotripsy and capturing the pieces of the kidney stone using the basket make procedures involving larger kidney stone relatively long in duration (e.g., exceeding two hours) and highly variable (e.g., risking complications involved in the extended presence of the ureteroscope within the patient). This is particularly true in lower-pole cases (e.g., cases involving stones located in the lower calyx of the kidney), which may be the most common kidney stone that patients incur. In such cases, due to the anatomical location of the kidney stone, the aforementioned lack of dexterity makes it extremely challenging to capture all stones with the basket effectively. Accordingly, in such cases patients may instead be referred to a more invasive procedure, such as percutaneous nephrolithotomy (PCNL) surgery. Of course, such issues are not only prevalent in ureteroscopic procedures, but throughout endoscopic procedures that address larger stones or other bodily objects in general.

To make ureteroscopy and, overall, endoscopy, more appropriate for the aforementioned burdens of larger stones or other bodily objects, it may be desirable to perform “aspiration” (e.g., suction-based) procedures, which may include drawing out the bodily objects en-masse using a vacuum source. However, the limited working channel size in conventional flexible endoscopes (one one-fifth millimeters) may be too small to be effective. Recently, a new class of modified bodily access sheaths have been developed to operate as suction catheters with large lumens, enabling suction and removal of stone fragments up to three millimeters in diameter. Suction through these modified bodily access sheaths has been shown to reduce operating time and improve stone free rates. This approach to stone removal may combine the advantages of dusting or basketing while substantially improving procedure times, especially for larger stones. In fact, aspiration procedures have been shown to result in better rates of “stone-free” (i.e. no stones remaining in the bodily region of interest) results than basketing, potentially saving patients from repeated operating room visits.

Despite significant promise of aspiration procedures, present systems present several barriers towards widespread adoption of the aforementioned bodily access sheaths acting as suction catheters. As an example in the ureteroscopic context, because of the size of present bodily access sheaths and the need for a large lumen through which to aspirate, the physician cannot use both a ureteroscope and a suction catheter at the same time using a trans-urethral approach. In this sense, the physician must fully remove the ureteroscope from the patient before putting the suction catheter in place, completely losing visualization and resulting in an increase in tool exchanges which could cause trauma along the ureter. The suction catheter must be placed into the ureter blindly by the physician and navigated to the correct site under continuous fluoroscopic feedback, exposing both the physician and the patient to harmful X-rays. As another example in the ureteroscopic context, present suction catheters have limited bi-directional deflection that is less than the ureteroscope itself (one-hundred and thirty-five degrees for the suction sheath, compared to two-hundred and seventy degrees for typical ureteroscopes). Thus, the physician must be careful to reposition kidney stones into more favorable locations within the kidney that are easily accessed by the less dexterous suction catheter. Of course, such issues are prevalent in not only the particular context of ureteroscopic procedures to remove kidney stones, but throughout the field of endoscopic removal of stones or other bodily objects.

What is needed, then, are improvements in devices and methods for stone aspiration procedures for ureteroscopic kidney stone removal within a patient.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One aspect of the present disclosure is a device for performing endoscopic surgery within a patient. The device includes a handle, a steerable sheath, a flexible tube, a light source, and an image sensor. The handle includes a passage. The steerable sheath is disposed on the handle. The steerable sheath includes a tip, a steerable section, and a lumen. The steerable section of the steerable sheath is actuable to form a bend, such that the tip is operable to be steered toward an anatomical region within a patient. The light source and the image sensor are disposed on the flexible tube. The flexible tube is configured to be inserted within the lumen and the inner passage, such that the light source and the image source are positioned at or near the tip of the steerable sheath. The flexible tube is further configured to be removed from the lumen, such that the lumen is operable to aspirate a bodily object from the anatomical region via the tip.

Another embodiment of the present disclosure is a method of performing endoscopic surgery within a patient. The method includes providing a steerable sheath, a flexible tube, a light source, and an image sensor. The steerable sheath includes a steerable section and a lumen. The flexible tube includes an end. The light source and the image sensor are disposed on the end of the flexible tube. The method further includes advancing the flexible tube through the lumen of the steerable sheath. The method further includes forming a bend in the steerable section of the steerable sheath. Forming the bend in the steerable section of the steerable sheath causes the tip to be steered toward an anatomical region within the patient. The method further includes retreating the flexible tube through the lumen, such that the lumen is open. The method further includes aspirating a bodily object through the lumen.

Another aspect of the present disclosure is a device for performing endoscopic surgery. The device includes a steerable sheath, a flexible tube, a light source, and an image sensor. The flexible tube includes an end. The steerable sheath includes tip, lumen, and a steerable section that is actuable to form a bend, such that the tip is operable to be steered toward an anatomical region within a patient. The steerable section includes a concentric tube structure having nested concentric tubes. Actuation of the steerable section is effectuated through applying an axial push-pull force to the concentric tubes. The light source and the image sensor are disposed on the end of the flexible tube. The flexible tube is disposed within the lumen such that the end is positioned at or near the tip.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 is a perspective view of a device for endoscopic stone removal including a handle, a steerable sheath coupled to the handle, and an insert assembly inserted within the steerable sheath and the handle, according to some embodiments.

FIG. 2 is a detailed perspective view of a tip of the steerable sheath of FIG. 1, according to some embodiments.

FIG. 3 is a perspective view of the insert assembly of FIG. 1 being inserted into the steerable sheath and the handle of FIG. 1, according to some embodiments.

FIG. 4 is a perspective view of the insert assembly of FIG. 1, according to some embodiments.

FIG. 5 is a detailed perspective view of a flexible tube of the insert assembly of FIG. 4, according to some embodiments.

FIG. 6 is a perspective view of the steerable sheath and the handle of FIG. 1, according to some embodiments.

FIG. 7 is a detailed perspective view of the tip 16 of the steerable sheath of FIG. 6, according to some embodiments.

FIG. 8 is a side view of the steerable sheath of FIG. 1, according to some embodiments.

FIG. 9 is a side view of an outer sheath tube and an inner sheath tube of the steerable sheath of FIG. 8, according to some embodiments.

FIG. 10A is a side view of the steerable sheath of FIG. 8 being actuated to form a bend, according to some embodiments.

FIG. 10B is a perspective view of the steerable sheath of FIG. 8 being actuated to form a bend, according to some embodiments.

FIG. 11A is a side view of the steerable sheath of FIG. 8 being actuated to form a bend, according to some embodiments.

FIG. 11B is a perspective view of the steerable sheath of FIG. 8 being actuated to form a bend, according to some embodiments.

FIG. 12A is a detailed side view of the outer sheath tube of FIG. 9, according to some embodiments.

FIG. 12B is a detailed side view of the inner sheath tube of FIG. 9, according to some embodiments.

FIG. 13 is an alternative embodiment of the steerable sheath tube of FIG. 1

FIG. 14A is a side view of the steerable sheath tube of FIG. 13 being actuated to form a bend, according to some embodiments.

FIG. 14B is a perspective view of the steerable sheath tube of FIG. 13 being actuated to form a bend, according to some embodiments.

FIG. 15 is a detailed perspective view of a tip 16 of the steerable sheath of FIG. 13, according to some embodiments.

FIG. 16A is a perspective view of the device of FIG. 1 being operated to actuate the steerable sheath of FIG. 1, according to some embodiments.

FIG. 16B is a perspective view of the device of FIG. 1 being operated to actuate the steerable sheath of FIG. 1, according to some embodiments.

FIG. 17 is a schematic view of the device of FIG. 1, shown engaging a kidney stone within the anatomy of a patient, according to some embodiments.

FIG. 18 is a detailed schematic view of a steerable sheath and insert assembly of the device of FIG. 17, shown engaging a kidney stone within the anatomy of a patient, according to some embodiments.

FIG. 19 is a schematic view of the handle of FIG. 6 and the steerable sheath of FIG. 6, shown aspirating a kidney stone from within the anatomy of a patient, according to some embodiments.

FIG. 20 is a detailed schematic view of the steerable sheath of FIG. 19, shown aspirating a kidney stone from within the anatomy of the patient, according to some embodiments.

FIG. 21 is a perspective view of a device for endoscopic stone removal including a handle, a steerable sheath coupled to the handle, and an insert assembly affixed to the steerable sheath, according to some alternative embodiments.

FIG. 22 is a detailed perspective view of a tip of the steerable sheath of FIG. 21, according to some embodiments.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific device and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the device when in the orientation shown in the drawing. A person of skill in the art will recognize that the device can assume different orientations when in use.

The present disclosure provides devices and methods for endoscopic surgery within a patient. In particular, the present disclosure relates to retrieval and/or removal of bodily objects through aspiration during endoscopic (or, more particularly, ureteroscopic) procedures. For example, bodily objects that may be removed via the devices and methods described herein may include, but are not limited to, stones such as kidney stones or other calculi.

In particular, the present disclosure provides for an endoscope (or, in particular, a ureteroscope) device with a removable insert assembly that may be inserted within a steerable sheath, thereby providing a central core within the steerable sheath that is configured to deliver an image sensor, a light source, and/or one or more ports. The one or more ports may be used for endoscopic tool delivery (such as a laser lithotripsy device), irrigation (e.g., fluid delivery), and/or suction to aspirate fluid and/or other bodily objects. In other words, the present disclosure may provide for an endoscope device that initially acts as typical endoscopes do. However, when the insert assembly is removed from the steerable sheath, an open lumen within the steerable sheath is revealed that is configured for aspiration of stones or other bodily objects.

In some embodiments, the open lumen is larger than any of the one or more ports and is therefore more appropriately configured for aspiration of larger stones, stone fragments, or other bodily objects. Thus, for example, after a stone has been broken down into fragments using a laser lithotripsy device facilitated by the insert assembly, the insert assembly can be removed to leave the steerable sheath (and its now-revealed open lumen) directly at an anatomical region (e.g., a surgical site) for aspirating the fragments, thereby eliminating the need to blindly insert a separate suction catheter for aspirating the fragments and sparing both patients and physicians from harmful fluoroscopic radiation that is required for placement confirmation. Moreover, in addition or as an alternative to aspiration of stones or other bodily objects, the open lumen may serve as a conduit for delivery of additional tools (e.g. thermal ablation probes, baskets, biopsy tools, ultrasound imaging probes, surgical grasping tools, etc.) and/or for irrigation at the anatomical region.

Although often depicted herein as applied to ureteroscopic procedures for the removal of kidney stones from within a kidney (and the calyces located therein), it should be appreciated that the devices and methods described herein may be applied to a variety of endoscopic procedures for the removal of stones or other bodily objects within various anatomical settings, structures, and situations. For example, and as suggested above, endoscopes are used in many other areas of the body (e.g., the GI tract, the abdomen through natural orifice or other percutaneous approaches, the lung(s), the brain, etc.) to remove various sorts of bodily objects. Thus, it should be clear to one versed in the art that, in addition to applications directed to ureteroscopes and the removal of kidney stones, the present disclosure may be similarly useful for endoscopes and the removal of stones or other bodily objects in general. For example, it may be advantageous to provide a device that initially acts as current endoscopes do (e.g., delivering the aforementioned image sensor, light source, and/or one or more ports) and then, by removing the aforementioned insert assembly, transform the endoscope device into a steerable sheath with a lumen that is appropriately sized for aspiration of larger stones, stone fragments, or other bodily objects, as well as delivery of additional tools and/or irrigation at the anatomical region.

Overall, by combining conventional endoscopy or ureteroscopy procedures with laser lithotripsy and stone aspiration, the present disclosure may present advantageous solutions to the aforementioned dilemma associated with treating patients with larger stones or other bodily objects. As a non-limiting example, by resolving the aforementioned issues prevalent in ureteroscopy procedures, the present disclosure may render ureteroscopy the clear choice of treatment in cases of larger kidney stones, rather than more invasive means.

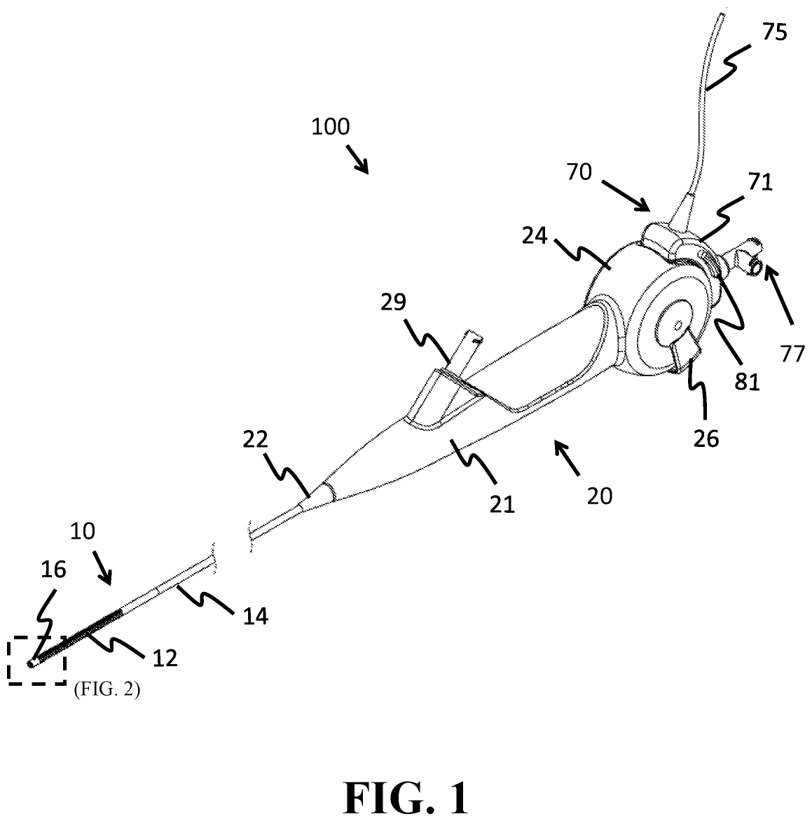

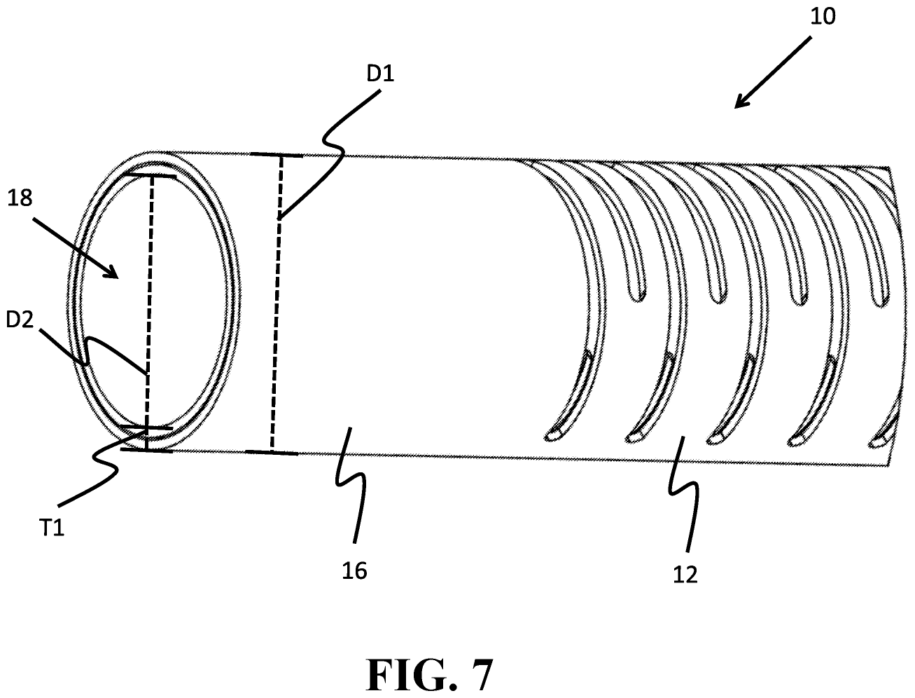

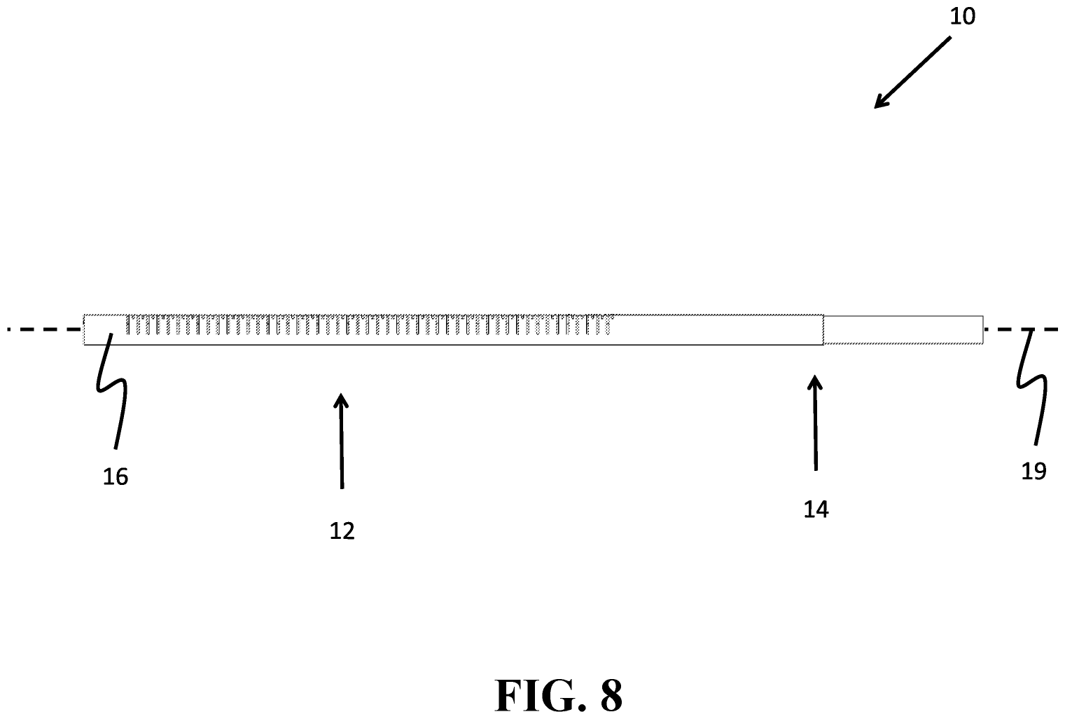

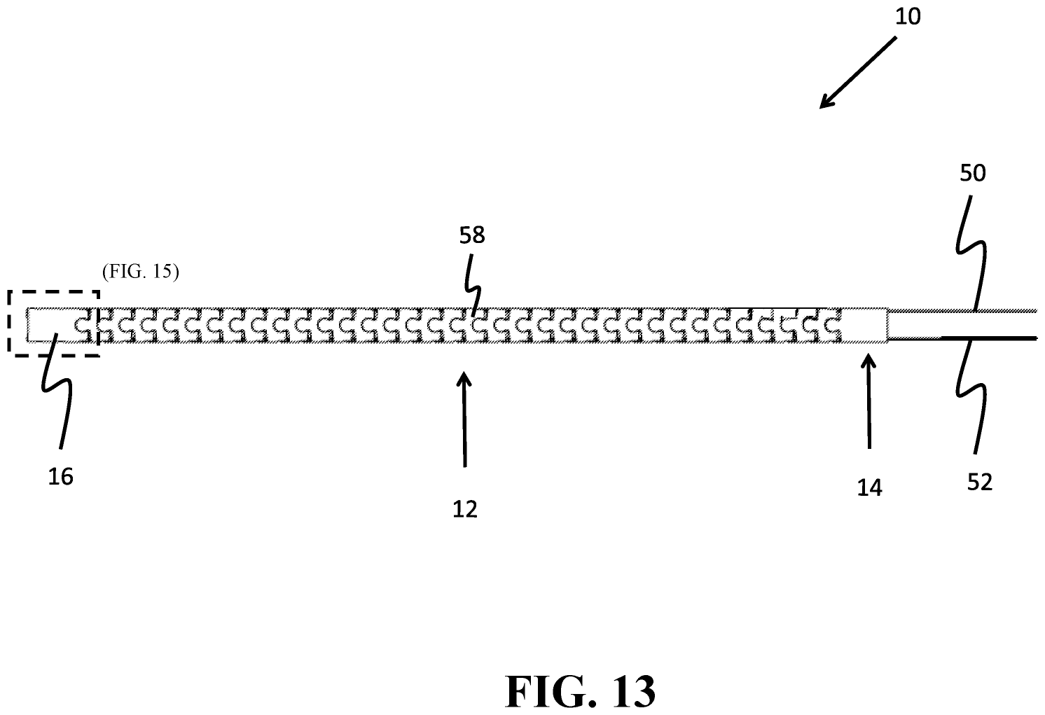

Referring now to FIGS. 1-3, an introductory embodiment of an device 100 for retrieving a bodily object from within a patient is generally illustrated. As described herein, the device 100 may provide a minimally-invasive medical device for removing bodily objects from within a patient. As a non-limiting example, in a ureteroscopic setting the device 100 may be configured to remove kidney stones from renal or ureteral systems of the patient. The device 100 may include a steerable sheath 10, an insert assembly 70, and a handle 20. The steerable sheath 10 may include a steerable section 12 and a transmission section 14. Further, the steerable sheath 10 may define an inner lumen 18 extending along the length of the steerable sheath 10. As described in greater detail below with reference to FIGS. 8-15, the steerable sheath 10 may be actuable to form a bend as operated by the handle 20, thereby deflecting a tip 16 located at a distal end of the steerable sheath 10 on a curved path. As described in greater detail below with reference to FIG. 7, the lumen 18 may define an outer diameter D1 that accommodates a first inner diameter D2 that is large enough for the aspiration of bodily objects from the anatomical region, deliverance of a first one or more endoscopic tools to the anatomical region, and/or irrigation of (e.g., providing irrigant flow to) the anatomical region. In some embodiments, the steerable sheath 10 is disposed on the handle 20. For example, the steerable sheet 10 may be mechanically coupled to the handle 20. As another example, the steerable sheath 10 and the handle 20 are formed as a single component.

In some embodiments, the insert assembly 70 includes a flexible tube 72 with an end 74. The flexible tube 72 may be configured to deliver various endoscopic components. For example, an image sensor 76 and a light source 78 may be disposed on the flexible tube 72. In some embodiments, the image sensor 76 and the light source 78 are disposed on the flexible tube 72. For example, the image sensor 67 and the light source 78 may be disposed on the end 74 of the flexible tube 72. In some embodiments, the flexible tube 72 includes a working channel 73, and/or an irrigation channel 79. The aforementioned endoscopic components, particularly the image sensor 76 and/or the light source 78, may be controlled via an insert assembly control module 71 in operable communication with the flexible tube 72.

In some embodiments, the insert assembly 70 may include a tool and/or irrigation port 77. As an example, the tool and/or irrigation port 77 may in communication with the irrigation flow channel 79 of the flexible tube 72 and be configured irrigate the anatomical region through the irrigation channel 79 at the end 74. As another example, the tool and/or irrigation port 77 may be in communication with the working channel 73 and be configured to receive a second one or more endoscopic tools (e.g., an appropriately sized device for laser lithotripsy procedures), which may be fed through the working channel 73 to end 74 for use at the anatomical region.

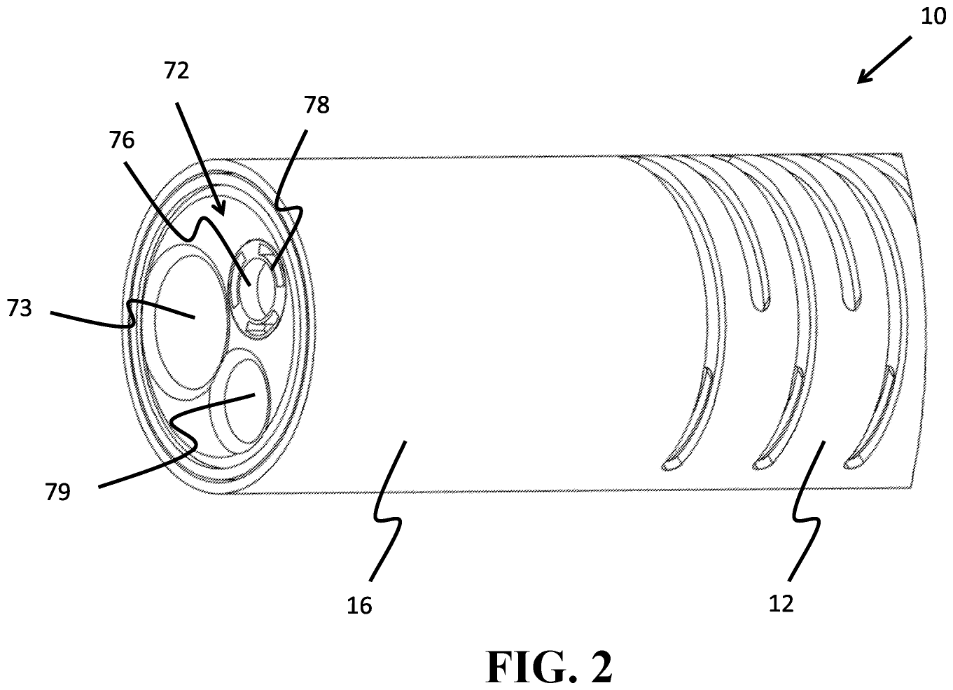

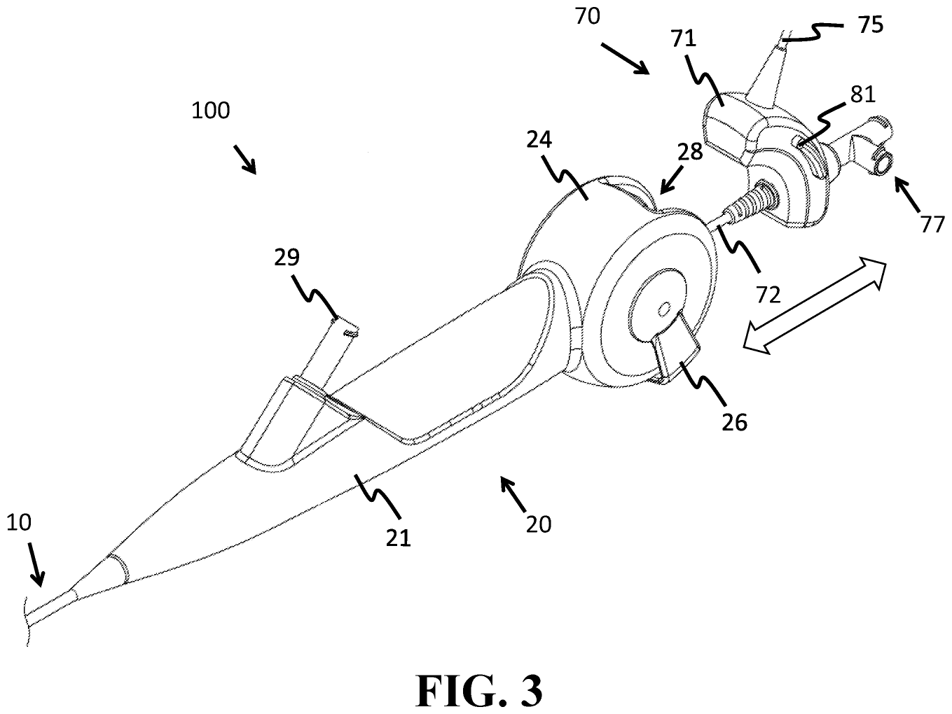

As shown with particular reference to FIG. 3, the insert assembly 70 may be inserted into the handle 20 at a proximal opening 28 of the handle 20, such that the insert assembly 70 is positioned within the an interior passage of the handle 20 and the lumen 18 of the steerable sheath 10, thereby providing various endoscopic devices at or near the tip 16. The flexible tube 72 may be advanced along the lumen 18 of the steerable sheath until the insert assembly 70 is fully seated within the handle 20 and the steerable sheath 10. As an example, when the insert assembly 70 is fully seated within the handle 20 and the steerable sheath 10, the control module 71 may be shaped to become seated within the proximal opening 28 of the handle 20. As another example, when the insert assembly is fully seated within the handle 20 and the steerable sheath 10, the end 74 of the flexible tube 72 may be positioned proximate the tip 16 of the steerable sheath 10. Thus, in the configuration shown with reference to FIGS. 1 and 2, the device 100 may provide a flexible endoscope or, more particularly a flexible ureteroscope

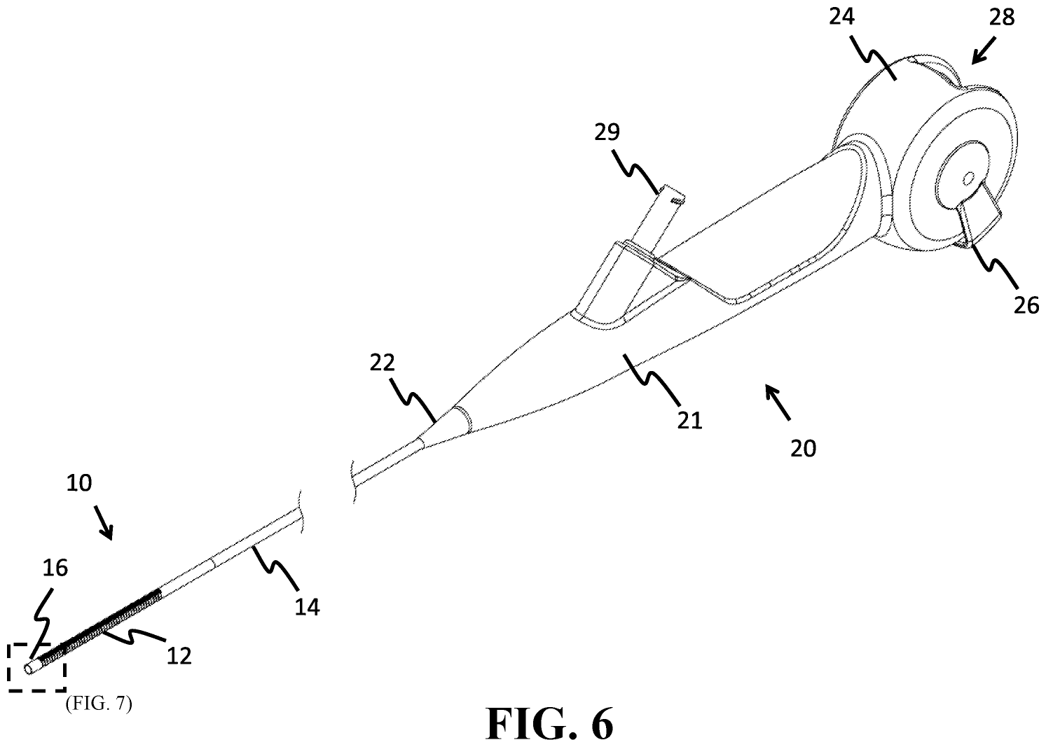

As shown with particular reference to FIG. 3, the insert assembly 70 may also be removed from the handle 20 and the steerable sheath 10 (e.g., by moving the insert assembly control module 71 out of, and away from, the proximal opening 28). For example, the flexible tube 72 may be retreated along the lumen 18, such that the lumen 18 is open. With respect to the steerable sheath 10 and the handle 20, this may result in a configuration depicted with reference to FIGS. 6 and 7. As mentioned above, the lumen 18 may be large enough for aspiration of bodily objects from the anatomical region. Thus, in some embodiments directed to ureteroscopic procedures, the steerable sheath 10 and the handle 20 as shown with reference to FIG. 3 are operable to perform kidney stone aspiration. In other embodiments, the steerable sheath 10 and the handle 20 as shown are operable to perform aspiration of other stones or bodily objects.

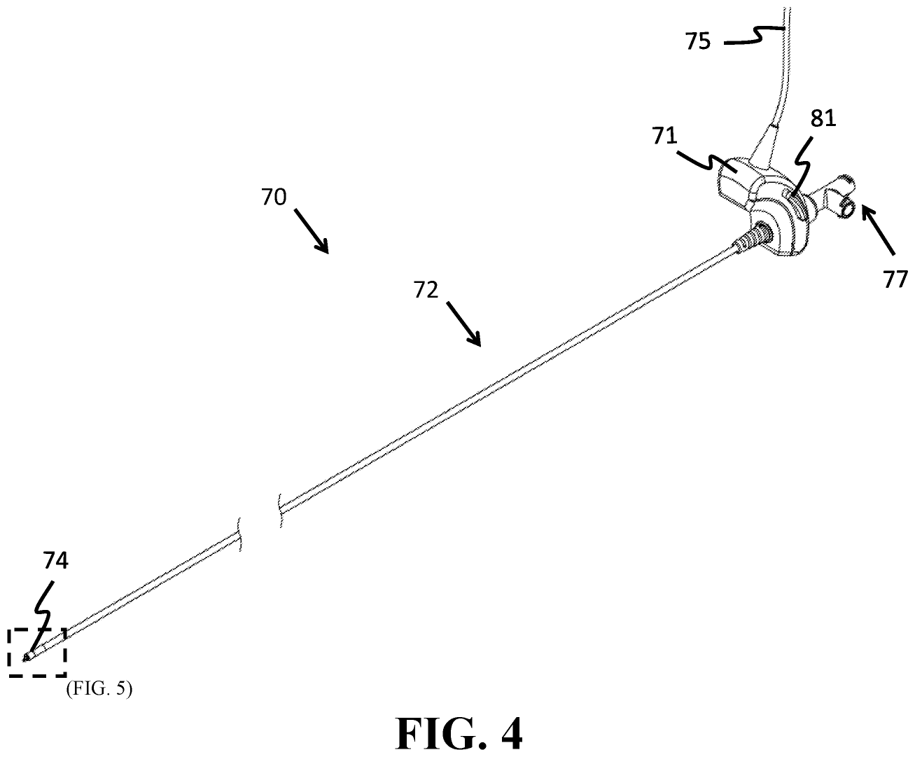

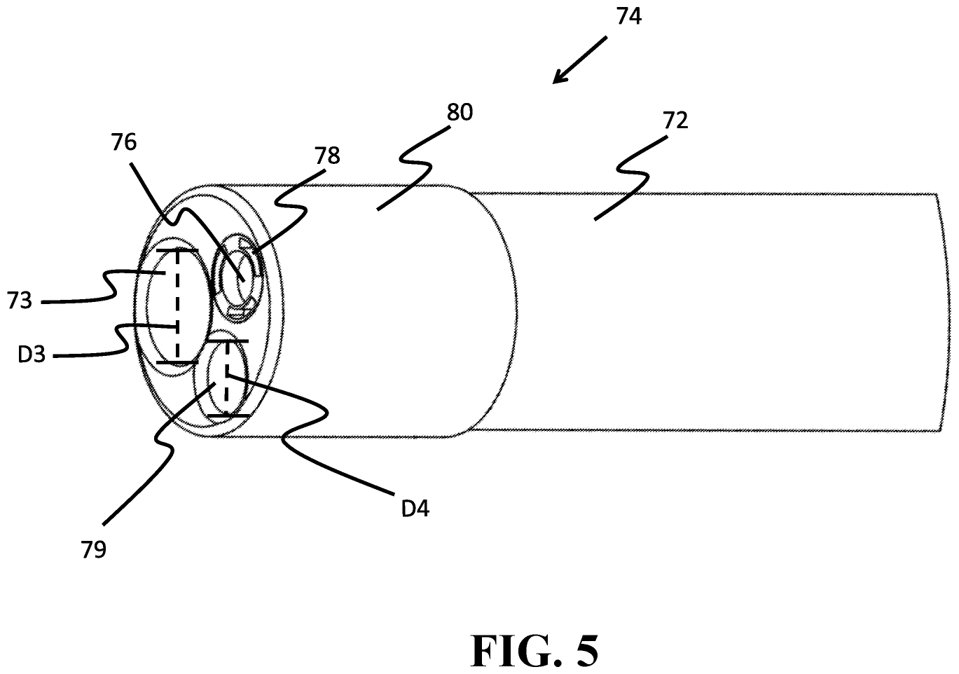

Referring now to FIGS. 4 and 5, the insert assembly 70 is shown, according to some embodiments. As mentioned above, the insert assembly 70 may include the flexible tube 72 which, as controlled by the insert assembly control module 71, facilitates the image sensor 76, light source 78, working channel 73, and/or irrigation channel 79. The flexible tube 72 may be passively flexible and house various wires and connections for operably coupling the image sensor 76 and the light source 78 to the insert assembly control module 71. As suggested above, the flexible tube 72 may house the working channel 73 and the irrigation channel 79, each of which terminates at the end 74 of the flexible tube 72. The working channel 73 may define a second inner diameter D3, and the irrigation channel 79 may define a third inner diameter D4. The second inner diameter D3 and the third inner diameter D4 may each be smaller than the first inner diameter D1 of the lumen 18. Although depicted herein as single channels, the working channel 73 and/or the irrigation channel 79 may include multiple channels, depending on the implementation.

In some embodiments, the image sensor 76, light source 78, irrigation channel 79, and/or working channel 73 are disposed on the end 74 via an endcap 80. The endcap 80 may be constructed in any suitable manner for providing the devices and methods described herein. As an example, the endcap 80 may be constructed of a biocompatible rigid plastic that has been injection molded. As another example, the endcap 80 may be 3D printed. As yet another example, the endcap 80 is machined via CNC or wire EDM techniques (using any number of biocompatible metal alloys that may be machined into a desired configuration). In some embodiments, the endcap 80 includes a keyway feature that engages with a corresponding keyed feature on the steerable sheath 10 in order to prevent rotation of the flexible tube 72 and/or the steerable sheath 10 relative to one another.

In some embodiments, the image sensor 76 includes a digital complementary metal oxide semiconductor (CMOS) sensor for providing digital visualization of an anatomical region. Via the CMOS sensor, the image sensor 76 may thus provide the required resolution, frame rate, and field-of-view to provide high quality imaging of the anatomical region. Additionally, the image sensor 76 may include optical pathways (e.g., lenses, filters) to precondition incoming light into an optimal format for image detection by the CMOS sensor. The image sensor 76 may be contained within a separate housing (alone or within the endcap 80, for example) designed to provide mechanical fixation to the flexible tube 72, light source 78, and/or the working channel 73. Signals to and from the image sensor 76 may be transmitted via cabling that extends along the length of the flexible tube 72 into the insert assembly control module 71. In alternative embodiments, the image sensor 76 is located away from the end 74. For example, the image sensor 76 may include fiber optics cables extending through the flexible tube 72 to the distal tip 54 and relaying images back to the image sensor 76. Accordingly, the image sensor 76 may be located in various locations on the device 100, such as the insert assembly control module 71 or any other suitable location.

In some embodiments, the light source 78 is a bundle of glass fiber optics for transmitting light from the insert assembly control module 71 to the end 74, in order to provide illumination to the anatomical region. For example, the light source 78 may include fiber optics cables extending through the flexible tube 72 to the distal tip 54. Such fiber optics cables may be configured to transmit light from a lamp (LED or incandescent, as examples). The lamp, in turn, may be disposed on the insert assembly control module 71 or the external video processor unit. Such fiber optics cables may be constructed to prevent breakage and light leakage at the minimum radius of curvature expected to be encountered during normal use of the flexible tube 72.

As suggested above, the image sensor 76 and/or the light source 78 may be controlled via the insert assembly control module 71. For example, the insert assembly control module 71 may include various programmable buttons (such as a button 81) or other interfaces operable to provide trigger signals to the image sensor 76 and/or the light source 78. As examples, the button 81 may be pressed by a user in order to activate or deactivate the light source 78, implement an image capture function on the image sensor 76, implement a video recording feature on the image sensor 76, and so on. In some embodiments, the insert assembly control module 71 is powered by an external source. For example, the insert assembly control module 71 may be connected to an external power supply via a power supply line 75. In other embodiments, the insert assembly control module 71 is powered by an on-board battery. In other embodiments still, the insert assembly control module 71 is in electrical communication with the handle 20 when the insert assembly control module 71 is seated within the proximal opening 28, and may draw stored power from the power supply

As suggested above, the flexible tube 72 may be configured to connect the endcap 80 to the insert assembly control module 71, while providing an interior duct enabling the passage of requisite wires and/or fiber optics cables in order to facilitate the function of the image sensor 76, light source 78, and/or working channel 73. In preferred embodiments, the flexible tube 72 is long enough such that the end 74 (and/or the endcap 80) is positioned slightly within, is co-planar with, or projects at least slightly from the tip 16 of the steerable sheath 10 when the insert assembly 70 is assembled with the handle 20.

In some embodiments, the flexible tube 72 is constructed from a high-modulus polymeric material or metal alloy such as Nitinol. In such embodiments, the flexible tube 72 may feature a pattern of laser-cut or micro-machined slots (e.g., notches) employed to reduce the flexural compliance and modify the torsional and axial properties of the flexible tube 72. In other embodiments, the flexible tube 72 is a composite tube constructed from one or more layers (e.g., polymeric liner layers, polymeric jacket layers, braid reinforcement layers, some combination thereof, etc.). In preferred embodiments, the flexible tube 72 is configured to provide the necessary flexural compliance to enable the steerable tip 10 to fully deflect over its intended range of motion, and is configured to provide enough axial stiffness to prevent kinking when the insert assembly 70 is inserted within the handle 20.

In some embodiments, the mechanical properties of the flexible tube 72 are constant through the entire length of the flexible tube 72. In other embodiments, the mechanical properties of the flexible tube 72 vary along the length of the flexible tube 72. For example, depending on the clinical application of the device 100, it may be advantageous to configure a distal portion of the flexible tube 72 (e.g., toward the end 74) to have a lower bending stiffness than a proximal portion of the flexible tube 72 (e.g., toward the insert assembly control module 71). As mentioned above, the flexible tube 72 may feature a pattern of laser-cut slots. In such configurations, variation in mechanical properties along the length of the flexible tube 72 may be achieved by modulating the spacing (e.g., the pitch, the cut fraction, etc.) of the slots along the length of the flexible tube 72. As further mentioned above, the flexible tube 72 may be constructed from a composite tube constructed from one or more layers. In such configurations, variation in mechanical properties along the length of the flexible tube 72 may be achieved by modifying the jacket material or the braid reinforcement configuration along the length of the flexible tube 72.

Referring now to FIGS. 6 and 7, the handle 20 and the steerable sheath 10 are shown, according to some embodiments. The handle 20 may include a body 21, a steerable sheath control module 24 disposed on the body 21, a steerable sheath control member 26, the proximal opening 28 formed by the body 21, an aspiration port 29 disposed on the body 21, and a hub 22 disposed on the body 21 that couples the body 21 to the transmission section 14 of the steerable sheath 10. As mentioned above, the configuration shown may be provided by removing the insert assembly 70 from the steerable sheath 10 and the handle 20 (e.g., by moving the insert assembly control module 71 out of, and away from, the proximal opening 28), leaving the lumen 18 open, such that the steerable sheath 10 and the handle 20 may be operable to perform aspiration procedures. As a corollary, in some embodiments, the proximal opening 28 is be configured to enable insertion of the insertion assembly 70 within the handle 20 and the steerable sheath 10, thereby assembling the device 100 shown above with reference to FIG. 1.

In some embodiments, the lumen 18 is configured to define the first inner diameter D2 such that the lumen 18 is appropriate for the aspiration of stones (kidney stones, in ureteroscopic contexts), stone fragments, and other bodily objects when the insert assembly 70 is removed as described herein. Moreover, the lumen 18 may be configured to irrigate the anatomical region or deliver the aforementioned first one or more endoscopic tools to the anatomical region. As an example, the lumen 18 may be configured to provide greater irrigation than the third inner diameter D4 of the irrigation channel 79 may allow. As another example, the first one or more endoscopic tools may be larger than the aforementioned second one or more endoscopic tools that are appropriate for delivery via the working channel 73. In this sense, the first one or more endoscopic tools may include lasers for laser lithotripsy procedures, thermal ablation probes, baskets, biopsy tools, ultrasound imaging probes, surgical grasping tools, and so on, any of which may not be appropriate for deliverance to the anatomical region via the working channel 73. In this sense, of course, the first inner diameter D2 may be larger than the second inner diameter D3 and the third inner diameter D4.

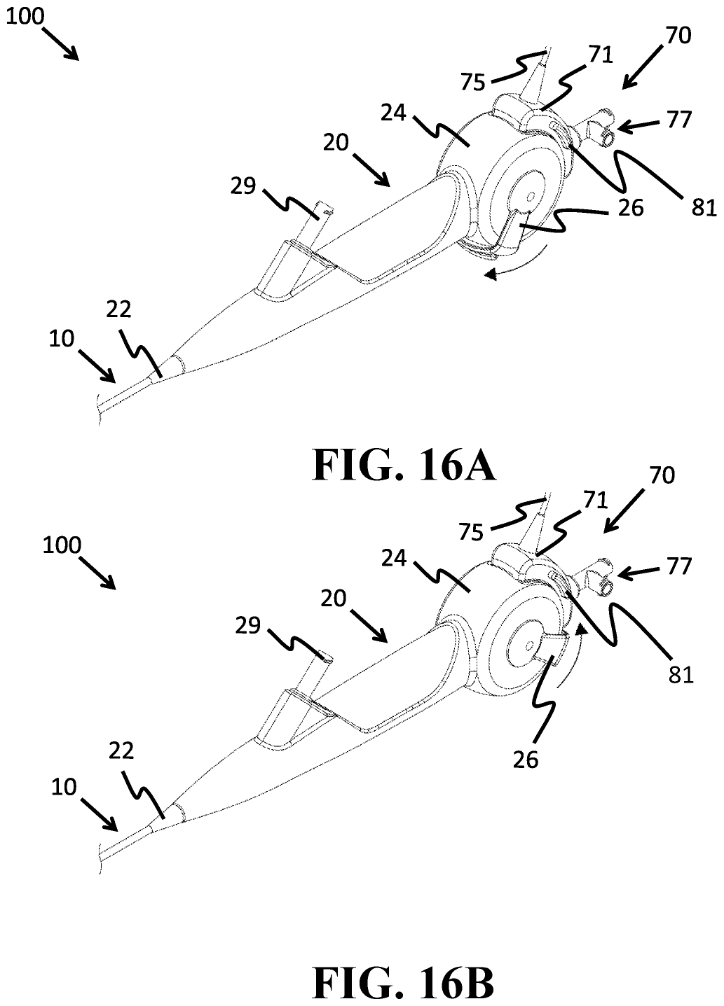

In some embodiments, the handle 20 includes various features to enable a user to control the steerable sheath 10. For example, the steerable sheath control member (e.g., a lever, switch, knob, button, etc.) 26 may be disposed on the steerable sheath control module 24 and may be manipulated by a user (as shown with reference to FIGS. 16A and 16B) in order to actuate the bending of the steerable section 12 of the steerable sheath 10 (as described in greater detail below with reference to FIGS. 8-15). In particular, the steerable sheath control member 26 may be in operable communication with a mechanical transmission disposed on or within the body 21. The mechanical transmission may be configured to convert manipulation of the steerable sheath control member 26 into various mechanical engagements with the steerable sheath 10 in order to actuate the bending of the steerable section 12.

In some embodiments, operation of the mechanical transmission and the steerable sheath control member 26 is supported by a power supply. For example, manipulation of the steerable sheath control member 26 may, by a controller on the steerable sheath control module 24, be converted to an electrical signal that is communicated to the mechanical transmission which, in turn, converts the electrical signal to the appropriate mechanical engagements with the steerable sheath 10. In other words, the mechanical transmission may operate as an electric motor. As such, a power supply may be used to facilitate the electrical signal of the steerable sheath control module 24 and the function of the mechanical transmission when operating as an electric motor. Accordingly, the steerable sheath control module 24 (or, generally, the handle 20) may include a power supply. In some embodiments, the power supply is external (e.g., a wired connection). In other embodiments, an on-board battery is stored on the steerable sheath control module 24 or elsewhere on the handle 20, thereby providing a power supply.

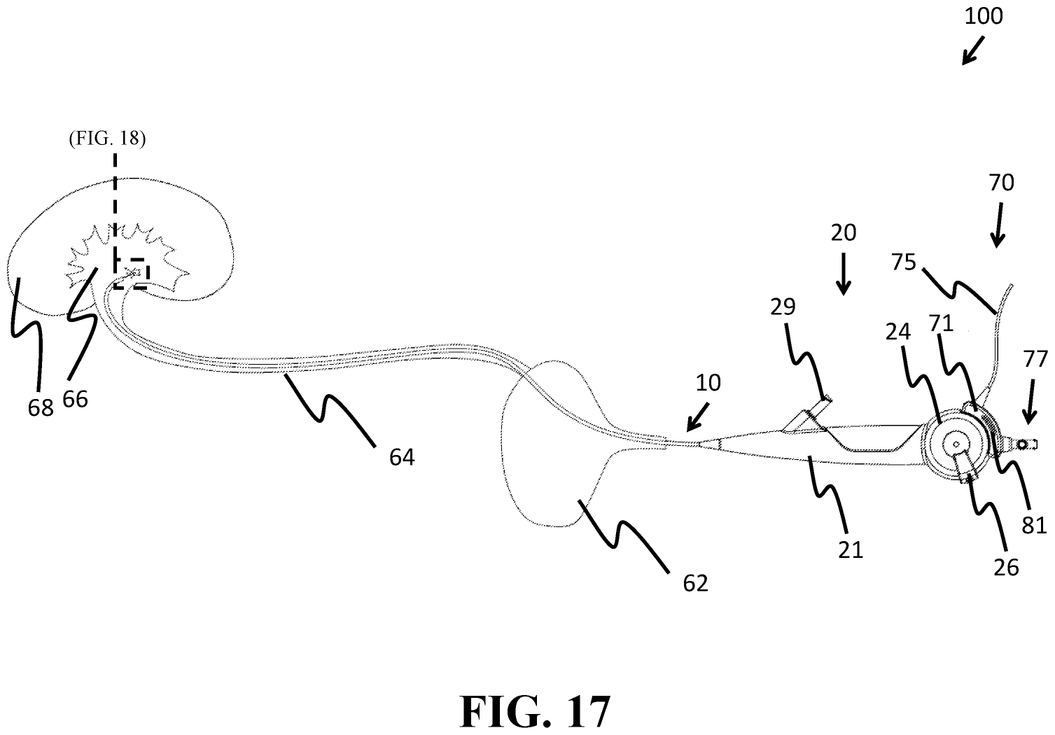

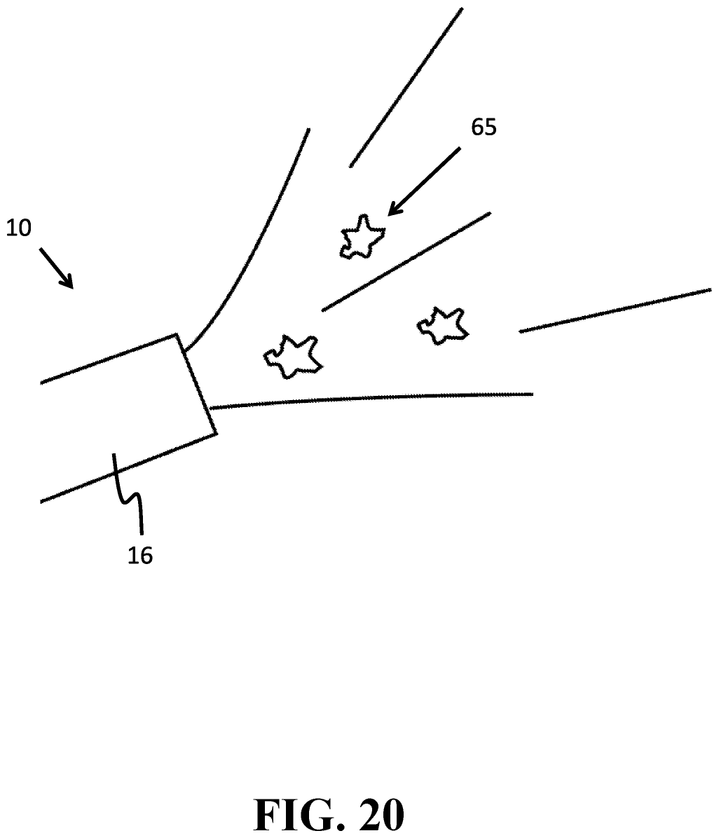

As mentioned above, the steerable sheath 10 includes the transmission section 14, which couples the steerable section 12 to the handle 20 at the hub 22. The transmission section 14 may incorporate the requisite flexural compliance, torsional stiffness, and axial stiffness required to navigate the expected patient anatomy during a clinical procedure. For example, as shown with reference to FIGS. 17-20, this may include travel through the bladder, up the ureter, and into the kidney.

Any number of manufacturing techniques common to the fabrication of flexible medical devices may be used to manufacture the flexible transmission section 14, so long as the flexible transmission 14 exhibits the requisite mechanical properties (e.g., flexural compliance, axial stiffness, torsional stiffness, minimum radius of curvature). In some embodiments, the transmission section 14 is an extension of the same material used to construct the steerable section 12 as discussed below. In other embodiments, the transmission section 14 may be constructed of a separate material that is attached to the material of the steerable section 12 via any number of fixation methods including, but not limited to, reflow, laser welding, adhesive, or mechanical fixation methods such as crimping, keying or swaging. In some embodiments, the transmission section 14 is constructed from a high-modulus polymeric material or metal alloy such as Nitinol. In such embodiments, the transmission section 14 may feature a pattern of laser-cut or micro-machined slots employed to reduce the flexural compliance and modify the torsional and axial properties of the transmission section 14. In other embodiments, the transmission section 14 is a composite tube constructed from one or more layers (e.g., polymeric liner layers, polymeric jacket layers, braid reinforcement layers, some combination thereof, etc.).

In some embodiments, the mechanical properties of the transmission section 14 are be constant through the entire length of the transmission section 14. In other embodiments, the mechanical properties of the transmission section 14 vary along the length of the transmission section 14. For example, depending on the clinical application of the device 100, it may be advantageous to configure a distal portion of the transmission section 14 (e.g., toward the sheath tip 16) to have a lower bending stiffness than a proximal portion of the transmission section 14 (e.g., toward the handle 20). As mentioned above, the transmission section 14 may feature a pattern of laser-cut slots. In such configurations, variation in the mechanical properties along the length of the transmission section 14 may be achieved by modulating the spacing (e.g., the pitch, the cut fraction, etc.) of the slots along the length of the transmission section 14. As further mentioned above, the transmission section 14 may be constructed from a composite tube constructed from one or more layers. In such configurations, variation in mechanical properties along the length of the transmission section 14 may be achieved by modifying the jacket material or the braid reinforcement configuration along the length of the transmission section 14.

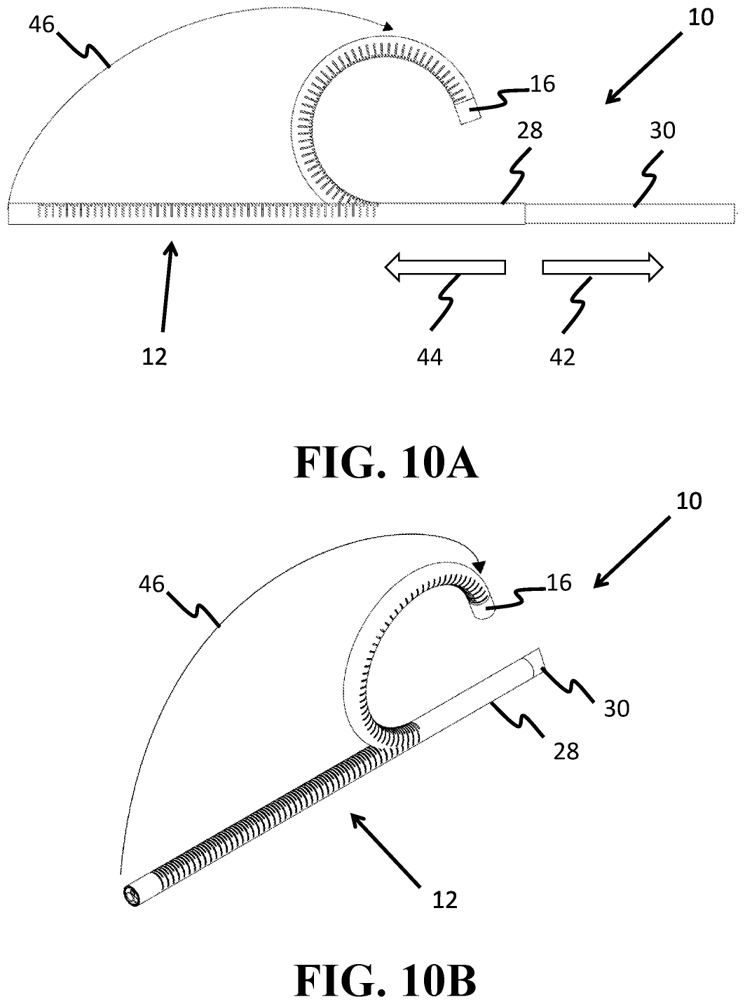

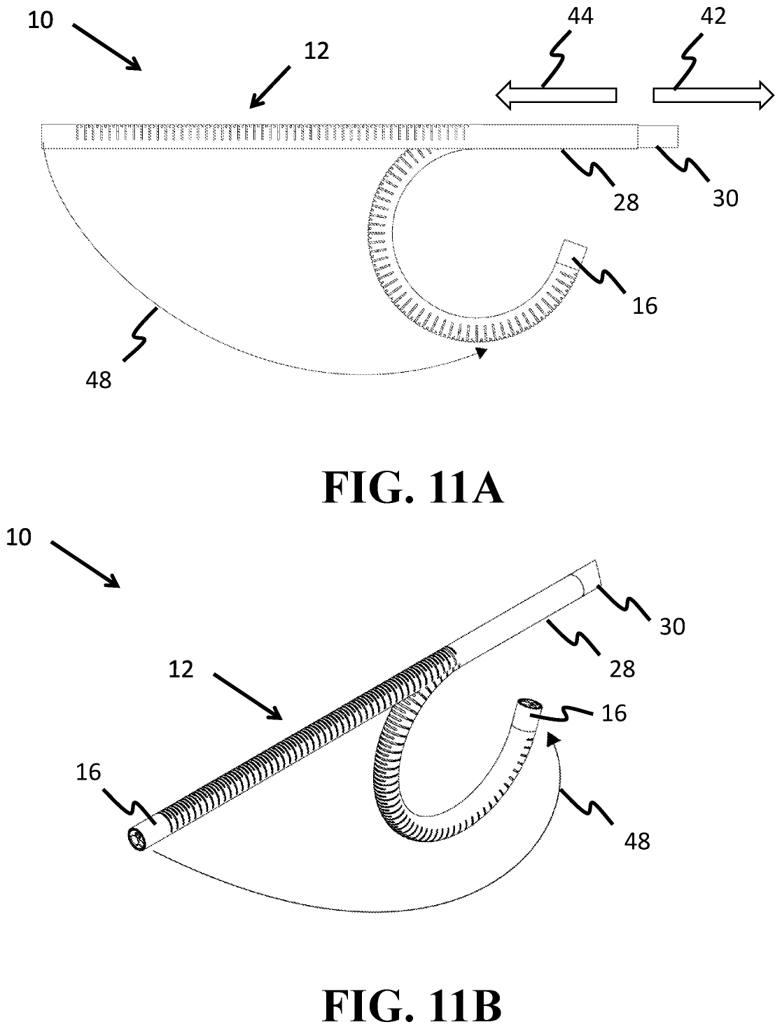

Referring now to FIG. 8, the steerable sheath 10 is shown, according to some embodiments. As mentioned above, the steerable sheath 10 includes the steerable section 12 and the transmission section 14. In particular, the steerable section 12 may be configured to generate an amount of angulation (e.g., bend at a bi-directional angle) relative to a geometric (e.g., central) axis 19 defined by the steerable sheath 10 (when arranged in a straight or linear configuration), thereby deflecting the tip 16 along a path of curvature. For example, with reference to FIGS. 10A-11B, the tip 16 may be deflected along a first path of curvature 46 or a second path of curvature 48, as described in greater detail below. This may allow the tip 16 to be steered toward an anatomical region within a patient (e.g., towards the anatomical region, towards the bodily object, toward the stone or kidney stone, etc.). Depending on the implementation, the steerable section 12 may exhibit a constant curvature when the tip 16 is deflected, or the steerable section 12 may exhibit a variable curvature.

As suggested above, depending on the implementation, various properties of the steerable sheath 10 may be configured in accordance with a clinical procedure that the device 100 is provided for. As a first example, the steerable section 12 may be configured to bend relative to the longitudinal axis 18 as required by a particular clinical procedure. As a second example, the steerable section 12 may be configured to generate an appropriate radius of curvature relative to the longitudinal axis 18 (e.g., when the tip 16 is deflected in accordance with the manipulation of the steerable sheath 10) for the particular clinical procedure. As a third example, the outer diameter D1 formed by the steerable sheath 10 may be appropriately sized for the particular natural orifice associated with the particular clinical procedure. As a fourth example, the transmission section 14 may be long enough to deliver the tip 16 on the steerable section 12 to an anatomical region associated with the clinical procedure (via a method of insertion into the body as shown with reference to FIGS. 17-20). As a fifth example, a length of the steerable section 12 may be defined according to the particular natural orifice associated with the particular clinical procedure or, in other words, the aforementioned bending and radius of curvature appropriate for the particular clinical procedure. As a sixth example, the steerable sheath 10 may have a wall thickness T1 required to house the flexible tube 72 within the lumen 18, while also being operable to aspirate stones (kidney stones, in some cases), stone fragments, or other bodily objects through the lumen 18 when open.

As a non-limiting example, the steerable sheath 10 may be configured for a flexible ureteroscopy procedure. In such configurations, the steerable section 12 may be arranged to bend to a bi-directional angle of between one-hundred and eighty and two-hundred and seventy degrees relative to the longitudinal axis 18; the steerable section 12 may be arranged to provide a radius of curvature of between fifteen and thirty millimeters; the outer diameter D1 of the steerable sheath 10 may be between two and three-tenths millimeters and four millimeters; the length of the transmission section 14 may be between about seven-hundred and eight-hundred millimeters; the length of the steerable sheath 10 may be long enough to enable the aforementioned bi-directional angle with the aforementioned radius of curvature; and the wall thickness T1 may be between about one twentieth millimeters and one fifth millimeters.

In some embodiments, the steerable sheath 10 (or the steerable section 12 in particular, in some cases) includes a jacket along the outside of the steerable sheath 10. In some arrangements, the jacket may be configured to provide a lubricious outer-coating to facilitate insertion into one or more natural orifices for various clinical procedures. In some arrangements, the jacket may be configured to provide clearance between the jacket itself and the outer diameter D1 of the steerable sheath 10. In this sense, the jacket may be configured to allow a passage of irrigant flow between the jacket and the outer surface of the steerable sheath 10. Advantageously, such a configuration may be useful for washing away debris or modulating intrarenal pressure. In some embodiments, the steerable sheath 10 includes a radio opaque marker to enable visualization via an external fluoroscopic imaging modality.

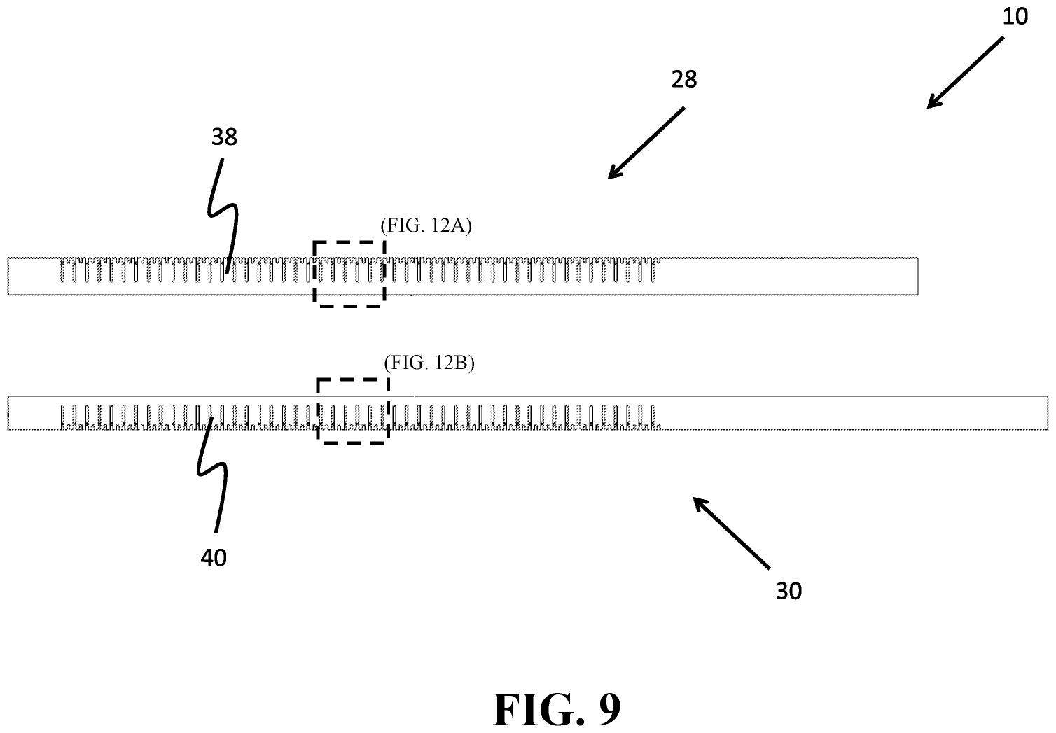

Referring now to FIGS. 9-12B, an example implementation of the steerable sheath 10 is illustrated, according to some embodiments. As described in greater detail below, the steerable sheath 10 may include a concentric tube structure having concentric nested tubes, and actuation of the steerable section 12 may be effectuated through applying an axial “push”-“pull” force to the concentric tubes. In other words, actuable bending of the steerable section 12 of the steerable sheath 10 (thereby deflecting the tip 16) may be provided through an agonist-antagonist concentric tube actuation configuration. In such configurations, the steerable sheath 10 includes an outer tube 30 and an inner tube 28 disposed within the outer tube 30. Each of the inner tube 28 and the outer tube 30 may define an outer dimension of between fifty and two-hundred micrometers in diameter. For instance, the inner tube 28 and the outer tube 30 may be configured so as to provide the lumen 18 in a configuration that defines the first inner diameter D2 such that the first inner diameter D2 is appropriate for the aspiration of stones, stone fragments, or other bodily objects when the insert assembly 70 is removed. In some arrangements, the inner tube 28 and the outer tube 30 are constructed of super-elastic Nitinol. In other arrangements, the inner tube 28 and the outer tube 30 are made of some other suitable material including, but not limited to, Polyimide, PEBAX, Nylon12, HDPE, LDPE, or any number of clinical grade, biocompatible thermoplastics or thermosets.

In such agonist-antagonist configurations, the steerable sheath 10 may implement an asymmetrical stiffness between the inner tube 28 and the outer tube 30. In other words, the inner tube 28 and the outer tube 30 may be configured to exhibit a first neutral axis 32 and a second neutral axis 34 (as shown with reference to FIGS. 10A and 10B) that are different and each offset from the geometric axis 19 of the steerable portion 12 of the steerable sheath 10. The asymmetrical stiffness between the inner tube 28 and the outer tube 30 may allow for actuable bending (on a singular plane or multiple planes) along the steerable portion 12. The inner tube 28 and the outer tube 30 may be coupled at their tips (e.g., at the tip 16), and rotationally aligned such that the first neutral axis 32 and the second neutral axis 34 oppose each other. Thus, when a differential force is applied to the inner tube 28 and the outer tube 30, the steerable section 12 may bend bi-directionally, depending on the differential force.

As an example of the differential force for such agonist-antagonist configurations, the inner tube 28 may be “pulled” in a first axial direction 42 without applying a corresponding force to the outer tube 30, thus causing the steerable section 12 to bend on a first path of curvature 46 as shown with reference to FIGS. 8A and 8B. As another example, the inner tube 28 may be “pushed” in a second axial direction 44 (opposite the first axial direction 42) without applying a corresponding force to the outer tube 30, thus causing the steerable section 12 to bend on a second path of curvature 48 as shown with reference to FIGS. 9A and 9B. As another example still, the aforementioned “push” and/or “pull” may be applied to the outer tube 30, rather than the inner tube 28. As yet another example, forces may be applied to each of the inner tube 28 and outer tube 30, such that the resulting force on the steerable sheath 10 is differential (e.g., the inner tube 28 is “pushed” while the outer tube 30 is “pulled,” the outer tube 30 is “pushed” while the inner tube 28 is “pulled”, the inner tube 28 and the outer tube 30 are each “pulled” to different degrees, the inner tube 28 and the outer tube 30 are each “pushed” to different degrees, and so on).

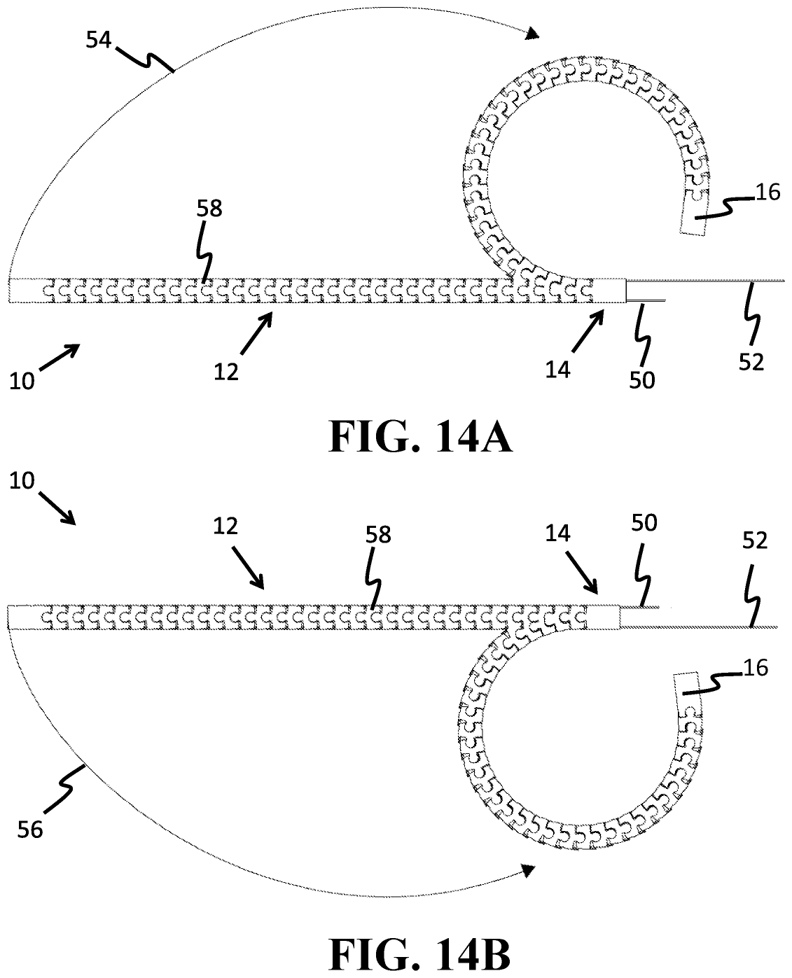

In some embodiments, the aforementioned “pushing” and/or “pulling” of the outer tube 30 and/or the inner tube 28 may be imparted by a mechanical transmission disposed on or within the handle 20. As described in greater detail below with reference to FIGS. 14A and 14B, the mechanical transmission may be operated by the steerable sheath control member 26.

In some embodiments associated with agonist-antagonist configurations, the asymmetrical stiffness is achieved by laser micro-machining a first pattern of cuts 38 into one side the inner tube 28, and a second pattern of cuts 40 into one side of the outer tube 30. The first pattern of cuts 38 and the second pattern of cuts 40 may reduce the bending stiffness of each of the inner tube 28 and the outer tube 30 (respectively). By providing the first pattern of cuts 38 and the second pattern of cuts 40 in an asymmetric fashion (e.g., at different “cut fractions,” at different “cut densities,” or otherwise at a different spacing along the length of the steerable portion 12), the associated reductions in bending stiffness properties of each of the inner tube 28 and the outer tube 30 may result in asymmetric stiffness between the inner tube 28 and the outer tube 30. In other words, the first pattern of cuts 38 on the inner tube 28 may result in the first neutral axis 32 offset from the central axis 36, and the second pattern of cuts 40 of on the outer tube 30 may result in the first neutral axis 34 that is different from the first neutral axis 32 and also offset from the central axis 36.

As mentioned above, the steerable portion 12 may exhibit a constant curvature when the tip 16 is deflected, or the steerable portion 12 may exhibit variable curvature. As an example of providing the steerable portion 12 in a configuration that exhibits variable curvature, the first pattern of cuts 38 and the second pattern of cuts 40 may be provided in a fashion of increasing density along the length of the first pattern of cuts 38 and the second pattern of cuts 40 toward the tip 16. In such an example, an overall stiffness of the steerable portion 12 may be reduced towards the tip 16, such that the steerable tip 16 is deflected in accordance with a distal rate of bending (associated with the a portion of the steerable section 12 that is closer to the steerable tip 16) that is greater than a proximal rate of bending (associated with the steerable section 12 closer to the transmission section 14). As another example, the first pattern of cuts 38 and the second pattern of cuts 40 may be provided in a fashion of decreasing density along the length of the first pattern of cuts 38 and the second pattern of cuts 40 toward the tip 16. In such an example, the overall stiffness of the steerable portion 12 may be increased towards the tip 16, such that the steerable tip 16 is deflected in accordance with a distal rate of bending (associated with the a portion of the steerable section 12 that is closer to the steerable tip 16) that is less than a proximal rate of bending (associated with the steerable section 12 closer to the transmission section 14).

In other embodiments associated with agonist-antagonist configurations, the asymmetrical stiffness between the inner tube 28 and the outer tube 30 is achieved by selective durometer variation between the inner tube 28 and the outer tube 30, at least with respect to portions of the inner tube 28 and the outer tube 30 that correspond to the steerable section 12. In this sense, through selective durometer variation in the inner tube 28 and the outer tube 30, the offset neutral axes such as the first neutral axis 32 and the second neutral axis 34 may be similarly provided, thereby effectuating the asymmetrical stiffness between the inner tube 28 and the outer tube 30.

In other embodiments associated with agonist-antagonist configurations still, the asymmetrical stiffness between the inner tube 28 and the outer tube 30 is achieved by selective ablation or integration of the material of the inner tube 28 and the outer tube 30. In such embodiments, the inner tube 28 and the outer tube 30 may be constructed as one or more layers (e.g., polymeric liner layers, polymeric jacket layers, braid reinforcement layers, some combination thereof, etc.), at least with respect to portions of the inner tube 28 and the outer tube 30 that correspond to the steerable section 12. As an example, through selective ablation of one or more layers of each of the inner tube 28 and the outer tube 30, the offset neutral axes such as the first neutral axis 32 and the second neutral axis 34 may be similarly provided. As another example, by integrating an axial portion of a layer (e.g., an axial braid member in cases of braid reinforcement layers) to be configured as a high-stiffness “backbone” on each of the inner tube 28 and the outer tube 30, the offset neutral axes such as the first neutral axis 32 and the second neutral axis 34 may be similarly provided.

Referring now to FIGS. 11-13 an alternative implementation of the steerable sheath 10 is illustrated, according to some embodiments. As described in greater detail below, the steerable sheath 10 may include a first pullwire (e.g., a cable) 50 and a second pullwire 52, each coupled to the tip 16 of the steerable sheath 10, and actuation of the steerable sheath 10 may be effectuated through applying a “pull” force to the first pullwire 50 or the second pullwire 52. In other words, actuable bending of the steerable section 12 of the steerable sheath 10 (thereby deflecting the tip 16) may be provided through pullwire actuation. The first and second pullwires 50, 52 may be positioned within the lumen 18 of the steerable sheath 10 and extend along the length of the steerable sheath 10. At one end, the first and second pullwires 50, 52 may be coupled to the tip 16 (via welding, adhesive, or mechanical crimping, for example). At the other end, the first and second pullwires 50, 52 may be coupled to a mechanical transmission disposed on or within the handle 20. As described in greater detail below with reference to FIGS. 16A and 16B, the mechanical transmission may be operated by the steerable sheath control member 26.

In some embodiments associated with pullwire actuation, the first and second pullwires 50, 52 are located within the lumen 18 at different circumferential positions in order to provide actuable bending as described below. For example, and as shown, the first pullwire 50 may be located on or near a first inner diametric end of the lumen 18, while the second pullwire 52 is located on or near another a second diametric end of the lumen 18 that is opposite the first diametric end. The first and second pullwires 50, 52 may be constrained within a liner tube inside of the steerable sheath 10 in order to prevent them from displacing laterally during actuation as described below. The first and second pullwires 50, 52 may be constructed of any suitable material that exhibits high tensile strength. As examples, the first and second pullwires 50, 52 may be constructed of Nitinol wire, extruded or braided stainless steel wire, and Kevlar.

In some embodiments associated with pullwire configurations, the mechanical transmission of the handle 20 may impart a differential tensile force on the first and second pullwires 50, 52. As an example, the mechanical transmission may impart a tensile force drawing the first pullwire 50 toward the handle 20, while leaving the second pullwire 52 stagnant, and vice-versa. As another example, the mechanical transmission may impart a first tensile force drawing the first pullwire 50 toward the handle 20, as well as a second tensile force drawing the second pullwire 52 toward the handle 20, such that the first tensile force is greater than or lesser than the second tensile force.

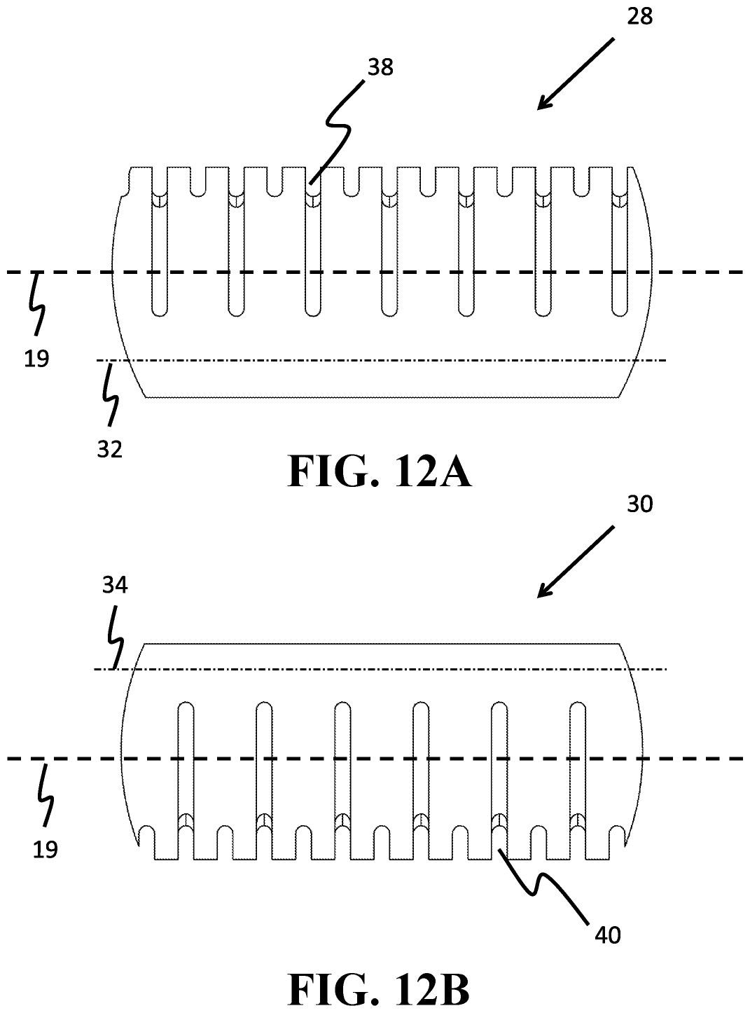

In some embodiments associated with pullwire configurations, the steerable section 12 of the steerable sheath 10 may be configured to exhibit anisotropic stiffness. For example, the steerable section 12 may be configured to exhibit a lower bending stiffness on one or more diametric portions of the steerable sheath 10 than other diametric portions. As depicted with reference to FIGS. 12A and 12B, the upper diametric end and lower diametric end of the steerable section 12 may exhibit a lower bending stiffness than the middle portions steerable section 12.

Due to the aforementioned anisotropic stiffness of the steerable section 12, as well as the different circumferential positions of the first and second pullwires 50, 52 within the lumen 18 as described above, when the mechanical transmission imparts the aforementioned differential force on the first and second pullwires 50, 52, the steerable section 12 may be actuated to bend. In particular, by positioning the first and second pullwires 50, 52 at opposite diametric ends of the lumen 18 where the steerable sheath 10 is configured to exhibit lower bending stiffness relative to other diametric portions, various differential forces may be applied to the first and second pullwires 50, 52 to provide for bi-directional bending of the steerable section 12 and deflection of the tip 16, as shown with reference to FIGS. 12A and 12B. As shown with particular reference to FIG. 12A, the mechanical transmission may impart a tensile force on the first pullwire 50, thereby causing the steerable section 12 to bend and deflecting the tip 16 along a first path of curvature 54. As shown with particular reference to FIG. 12B, the mechanical transmission may impart a tensile force on the second pullwire 52, thereby causing the steerable section 12 to bend and deflecting the tip 16 along a second path of curvature 56.

In some embodiments associated with pullwire configurations, and in order to facilitate the aforementioned anisotropic stiffness of the steerable section 12, the steerable section 12 may be formed as a serial linkage of tube segments 58 as shown. Due to the interlocking linkage between the tube segments 58, each tube segment may be configured to pivot about an axis defined by a previous tube segment (e.g., the tube segment further from the tip 16). As shown, each linkage defines the aforementioned axis in the same lateral direction, thereby promoting bi-directional bending of the steerable section 12 when actuated. In other such embodiments, the steerable sheath 10 is constructed with a polymeric material with a longitudinal braid. In other such embodiments, the steerable sheath 10 is constructed of metal (e.g., Stainless Steel, Nitinol, Titanium, etc.) that has been patterned with a series of laser-machined cuts. In various arrangements of pullwire configurations, the steerable sheath 10 may be disposed within an outer jacket.

As discussed above, the first and second pullwires 50, 52 may be provided in order to perform bi-directional bending of the steerable section 12 and deflection of the tip 16. In other words, the steerable section 12 as discussed above may bend on a single degree of freedom. In other embodiments, however, the steerable section 12 may be configured to bend on any number of degrees of freedom through the use of any number of pullwires. For example, the steerable sheath 12 may be actuated to bend on two-degree-of-freedom by providing a third pullwire and a fourth pullwire (in addition to the first and second pullwires 50, 52), resulting in four pullwires disposed within the lumen 18 and coupled to the tip 16 and the mechanical transmission of the handle 20. The four pullwires may be angularly separated by ninety degrees. In some arrangements associated with this example, the steerable section 12 may be provided with anisotropic stiffness that results in lower bending stiffness in four regions corresponding to the four pullwires. In other arrangements, the steerable section 12 may be provided via serial linkage as discussed above, however each linkage may allow each tube segment to pivot about two axes defined by the previous tube segment, rather than one, thereby promoting two degrees of freedom instead of one.

Referring now to FIGS. 14A and 14B, operation of the steerable sheath control member 26 of the handle 20 is shown, according to some embodiments. As mentioned above, the steerable sheath control member 26 of the handle 20 may be manipulated by a user in order to actuate the bending of the steerable section 12 of the steerable sheath 10. In particular, a mechanical transmission may be disposed on or within the handle 20, engage the steerable sheath 10, and be operated by the steerable sheath control member 26 in order to impart various forces on the steerable sheath 10, thereby actuating the steerable section 12. For example, the mechanical transmission may convert a deflection or translation of the steerable sheath control member 26 into motion of one or more components of the steerable sheath 10.

As mentioned above, actuable bending of the steerable section 12 of the steerable sheath 10 may be provided through an agonist-antagonist concentric tube actuation configuration. In such cases, the mechanical transmission, as operated by the steerable sheath control member 26, may be configured as a rack-and-pinion system, a slider crank mechanism, a leadscrew, or any combination of the above. As alternatively described above, actuable bending of the steerable section 12 of the steerable sheath 10 may be provided through pullwire actuation. In such cases, the mechanical transmission, as operated by the steerable sheath control member 26, may be configured as realized by a rack and pinion, pulley, or capstan system.

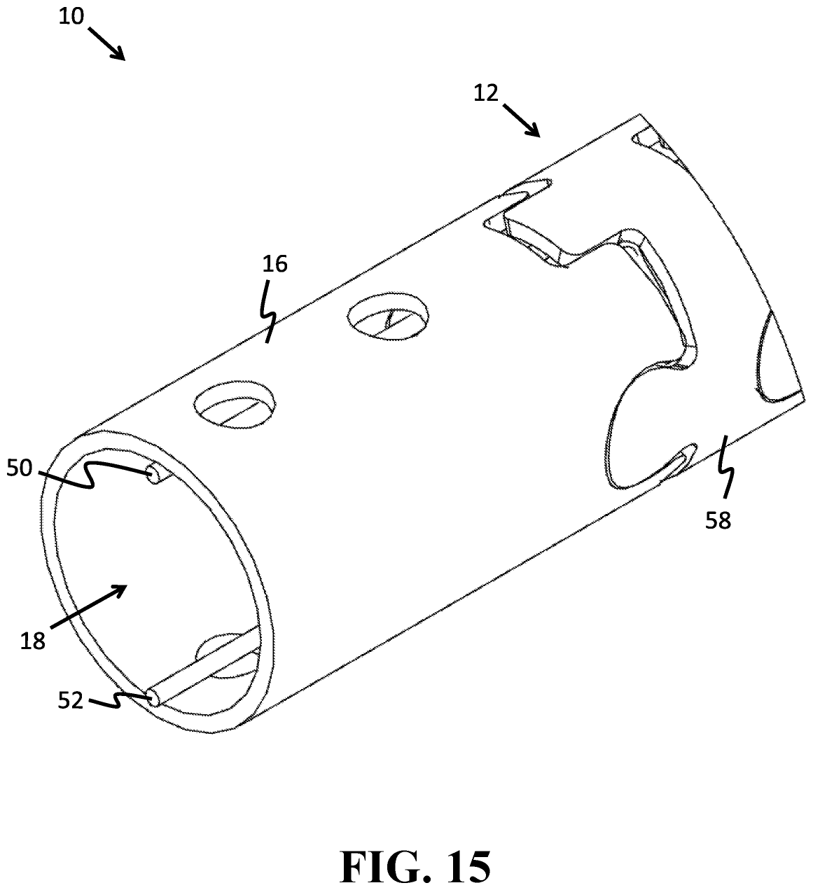

Referring now to FIGS. 15-18, an example utilization of the device 100 is shown, according to some embodiments. FIGS. 15-18 depict an exemplary utilization of the device 100 in a ureteroscopic context. However, as mentioned above, the devices and methods described herein may be applied to a wide variety of other endoscopic contexts. Accordingly, the depiction of the device 100 as shown with reference to FIGS. 15-18 should be considered non-limiting and otherwise exemplary in nature. As shown with particular reference to FIG. 15, the device 100 may include the insert assembly inserted into the handle 20 and the steerable sheath 10. Thus, the flexible tube 72 may be extended through the lumen 18 of the steerable sheath 10, such that the endoscopic devices facilitated by the flexible tube 72 (the image sensor 76, the light source 76, the working channel 73, and/or the irrigation channel 79) may interface with the tip 16 of the steerable sheath 10. Accordingly, the device 100 as shown may be operable as a flexible endoscope or, particularly, a flexible ureteroscope.

For example, as shown with particular reference to FIGS. 15 and 16, the steerable sheath 10 and the flexible tube 72 therein may be guided through a bladder 62 and a ureter 64 until the tip 16 of the steerable sheath is located within a calyces 66 of a kidney 68. In some cases, this may involve utilizing the image sensor 76 and light source 78 of the flexible tube 72 for visualization of the various bodily cavities, and/or actuating the steerable section 12 of the steerable sheath 10 to form various curvatures, thereby facilitating the guiding of the steerable sheath 10 and the flexible tube 72 therein. Once the tip 16 is located within the calyces 66, the image sensor 76, light source 78, and actuation of the steerable section 12 may be used to identify and guide the tip 16 towards a kidney stone 65, such that the kidney stone 65 may be engaged and removed from the calyces 66.

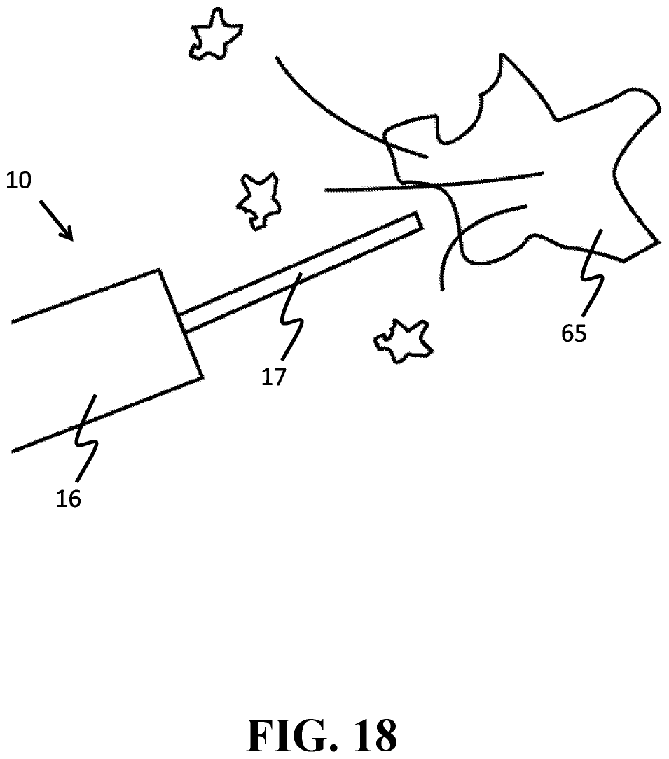

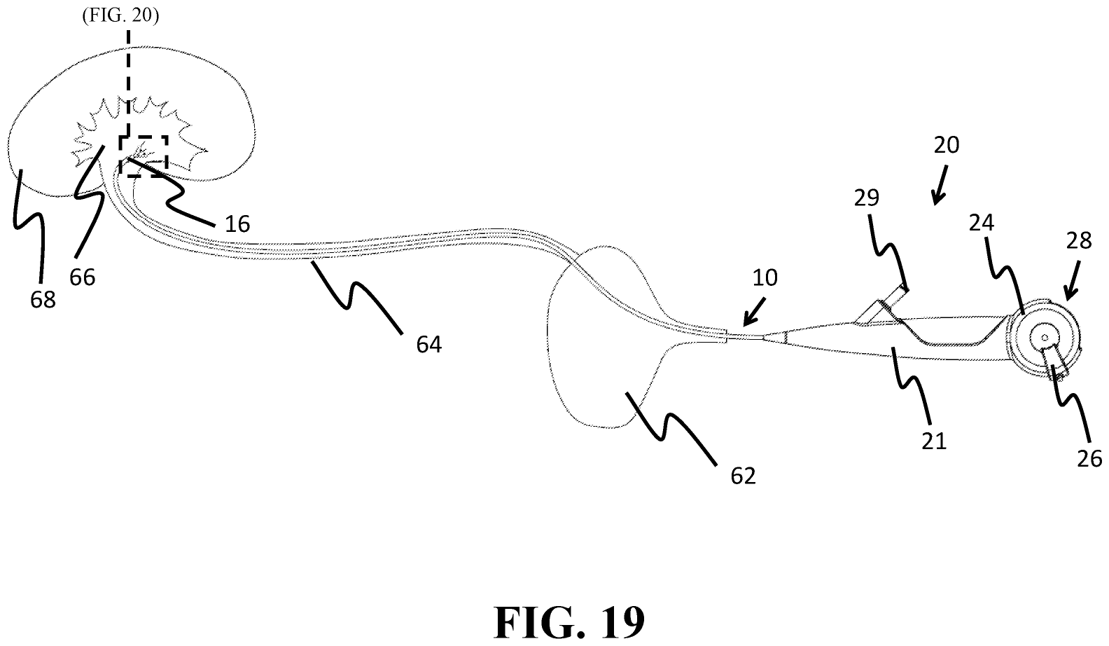

In some cases, the kidney stone 65 may be identified as being small enough for immediate aspiration, at which point the exemplary utilization of the device 100 may immediately proceed to the steps discussed below with reference to FIGS. 18 and 19. However if the kidney stone 65 is too large for immediate aspiration, a laser lithotripsy procedure may be conducted as discussed below with reference to FIGS. 16 and 17, before proceeding to aspiration of the kidney stone 65.

As shown with particular reference to FIG. 16, a laser device 17 may be extended from the working channel 73 of the flexible tube 72 in order to perform a laser lithotripsy procedure (e.g., breaking up the kidney stone 65 into smaller pieces). In some cases, the laser device 17 may be housed within the working channel 73 throughout the process of guiding the steerable sheath 10 and the flexible tube 72 towards the kidney stone 65. In other cases, the laser device 17 may be inserted into the tool and/or irrigation port 77 once the tip 16 has arrived at the kidney stone 65, guided through the working channel 73, and extended from the tip 16 as shown. Once the laser device 17 is engaged with the kidney stone 65 as shown, the laser device 17 may be controlled to break the kidney stone 65 into pieces that are small enough to be aspirated from the calyces 66 as shown with reference to FIGS. 18 and 19. At any preceding or ensuing point in time, irrigant may also be provided via the tool and/or irrigation port 77, such that it flows through the irrigation channel 79, and flows from the tip 16 in order to aid any of the aforementioned processes.

As shown with particular reference to FIGS. 18 and 19, once the kidney stone 65 has been broken up into small enough pieces via the laser lithotripsy procedure discussed above with reference to FIGS. 15 and 16 (or in cases where the kidney stone 65 was identified as being small enough for immediate aspiration as mentioned above), the insert assembly 70 may be removed from the steerable sheath 10 and the handle 20, thereby leaving the lumen 18 open in order to perform an aspiration procedure to removing the kidney stone 65 (or fragments thereof) from the calyces 66. For example, an external vacuum may be coupled to the aspiration port 29. The handle 20 may include various manifolds necessary for connecting the steerable sheath inner lumen 18 of the steerable sheath 10 to the aspiration port 29. When the external vacuum is activated to draw from the aspiration port 29, which is in communication with the lumen 18 of the steerable sheath 10, the kidney stone 65 (or fragments thereof) in the vicinity of the tip 16 may be drawn into the lumen 18 of the steerable sheath 10 via the steerable tip 16, travel along the lumen 18 of the steerable sheath 10, and be removed by the external vacuum via the aspiration port 29.

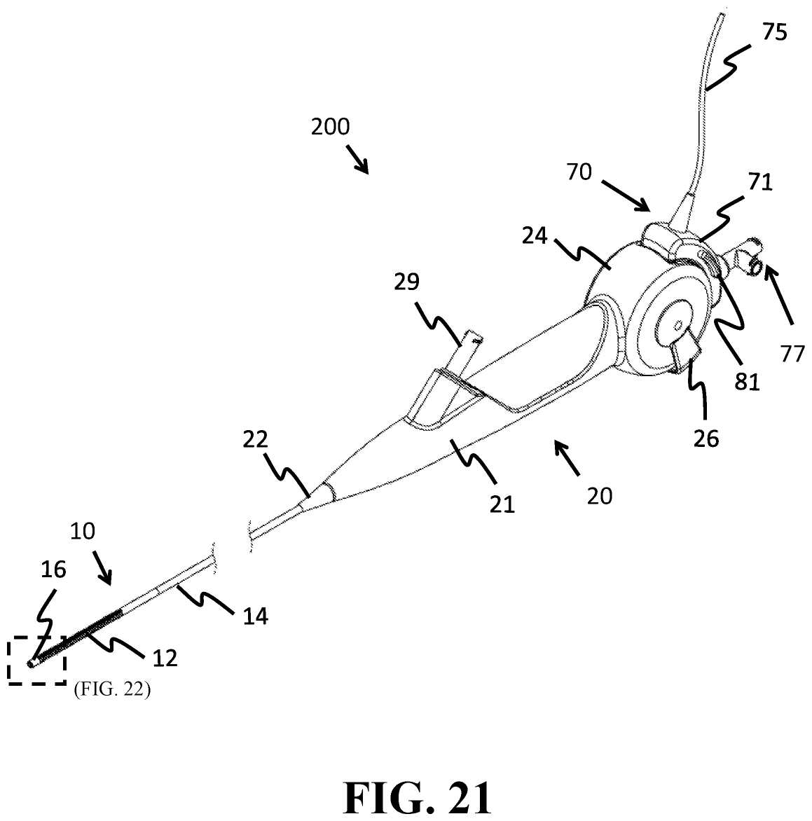

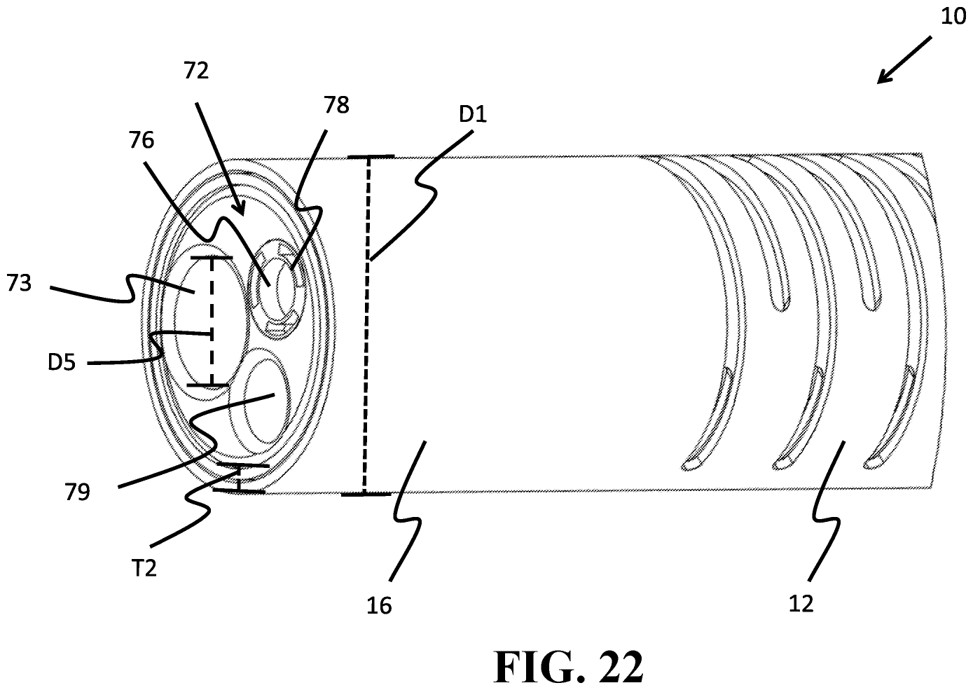

Referring again to FIGS. 21 and 22, an device 200 is shown, according to some alternative embodiments of the present disclosure. The device 200, like the device 100, may include the steerable sheath and the insert assembly 70 as well as, in some cases, the handle 20. In such alternative embodiments regarding the device 200, however, the insert assembly 70 may be permanently affixed to the steerable sheath 10 (and, in some cases, the handle 20). For example, as generally described herein in regards to the device 100, the insert assembly 70 may be inserted within the steerable sheath 10 and the handle 20 in order to provide a configuration that is operable as a utereroscope (e.g., by providing the image sensor 76, light source 78, working channel 73, and/or irrigation channel 79 at the tip 16 of the steerable sheath 10 via the flexible tube 72). The insert assembly 70 may then be removed from the steerable sheath 10 and the handle 20 in order to leave the lumen 18 of the steerable sheath 10 open for providing an operable aspiration configuration. However, in such alternative embodiments regarding the device 200 where the insert assembly 70 is permanently affixed to the steerable sheath 10, the device 100 may still be used to aspirate a kidney stone (e.g., without removal of the insert assembly 70 from the steerable sheath 10).

In such embodiments regarding the device 200 where the insert assembly 70 is permanently affixed, the steerable sheath 10 may include a concentric tube structure having concentric nested tubes, and actuation of the steerable section 12 may be effectuated through applying an axial “push”-“pull” force to the concentric tubes. As mentioned above with reference to FIGS. 9-12B, actuable bending of the steerable section 12 of the steerable sheath 10 (thereby deflecting the tip 16) may be provided through the described agonist-antagonist concentric tube actuation configuration.

In such embodiments regarding the device 200 where the insert assembly 70 is permanently affixed, the various tools facilitated by the flexible tube 72 may be fixedly attached at the tip 16 of the steerable sheath. For example, the endcap 80 disposed on the end 74 of the flexible tube 72 may be attached to the tip 16 of the steerable sheath 10. For example, the endcap 80 may be attached to the tip 16 by nesting the endcap 80 within the inner tube 28 of the steerable sheath 10 (in such agonist-antagonist concentric tube actuation configurations). As another example, the endcap 80 may be affixed to the inner tube 28, as well as the outer tube 30 in a butt-joint configuration. As other examples still, the endcap 80 may be affixed to the tip 16 method via biocompatible adhesive, mechanical crimping, welding or soldering. In other arrangements, the end 74 of the flexible tube is directly affixed to the tip 16 of the steerable sheath 10 (rather than via the endcap 80) using the aforementioned fixation methods.

In such embodiments regarding the device 200 where the insert assembly 70 is permanently affixed, the working channel 73 may be appropriately sized for providing kidney stone aspiration. For example, the working channel 73 may define fourth inner diameter D5 of about two millimeters or larger, thereby providing a viable channel for the deliverance of the aforementioned first one or more endoscopic tools, and/or the aspiration of stones or other bodily objects through the working channel 73. Further, the steerable sheath 10 may define a wall thickness T2 between about one twentieth millimeters and one fifth millimeters, as well as the outer diameter D1 between two and three-tenths millimeters and four millimeters. In this sense, the working channel 73 may be large enough to aspirate a kidney stone, while still being an appropriate size such that the flexible tube 72 may be facilitated by the steerable sheath 10 in navigating through the natural orifices associated with endoscopic or ureteroscopic procedures as discussed above. The working channel 73 may be in communication with an external vacuum via the aspiration port 29 or the tool/irrigation port 77, depending on the implementation.

Thus, although there have been described particular embodiments of the present invention of a new and useful METHOD AND DEVICE FOR ENDOSCOPIC ASPIRATION, it is not intended that such references be construed as limitations upon the scope of this invention.

What is claimed is:

1. A device for performing endoscopic surgery within a patient comprising:
a handle including a passage;
a steerable sheath disposed on the handle and including lumen, a tip, and a steerable section that is actuable to form a bend, such that the tip is operable to be steered toward an anatomical region within the patient;
a flexible tube including an end;
a light source disposed on the end; and
an image sensor disposed on the end,
wherein the flexible tube is configured to be inserted within the lumen and the passage, such that the light source and the image source are positioned at or near the tip, and
wherein the flexible tube is configured to be removed from the lumen, such that the lumen is operable to aspirate a bodily object from the anatomical region via the tip.

2. The device of claim 1, wherein the steerable sheath has a wall thickness of between about one twentieth millimeters and one fifth millimeters.

3. The device of claim 2, wherein the steerable sheath has an outer diameter of between about two and three tenths millimeters and about four millimeters.

4. The device of claim 1, wherein when the flexible tube is removed from the lumen, the lumen is further operable to deliver a first one or more tools to the anatomical region and irrigate the anatomical region.

5. The device of claim 1, wherein the flexible tube includes a working channel configured to deliver a second one or more tools to the anatomical region and an irrigation channel configured to irrigate the anatomical region, and
wherein a first inner diameter of the lumen is greater than each of a second inner diameter of the working channel and a third inner diameter of the irrigation channel.

6. The device of claim 1, wherein the handle further includes an aspiration port in communication with the lumen and an external vacuum device, such that the lumen is operable to aspirate a bodily object from the tip to the aspiration port.

7. The device of claim 1, wherein the steerable sheath includes a concentric tube structure having nested concentric tubes, and
wherein actuation of the steerable section is effectuated through applying an axial push-pull force to the concentric tubes.

8. The device of claim 1, wherein the steerable sheath includes a first pullwire and a second pullwire each coupled to the tip, and
wherein actuation of the steerable section is effectuated through applying a pull force to the first pullwire or the second pullwire.

9. A method for performing endoscopic surgery comprising:
providing a steerable sheath including a steerable section, a lumen, and a tip;
providing a flexible tube including an end;
providing a light source and an image sensor each disposed on the end;
advancing the flexible tube along an axis of the lumen, such that the light source and the image source are positioned at or near the tip;
forming a bend in the steerable section, wherein forming the bend causes the tip to be steered toward an anatomical region within a patient;
retreating the flexible tube along the axis, such that the lumen is open; and
aspirating a bodily object through the lumen via the tip.

10. The method of claim 9, wherein the steerable sheath has a wall thickness of between about one twentieth millimeters and one fifth millimeters, and
wherein the steerable sheath has an outer diameter of between about two and three tenths millimeters and about four millimeters.

11. The method of claim 9, further comprising:
delivering, via the lumen when the lumen is open, a first one or more tools to the anatomical region; and
irrigating, via the lumen when the lumen is open, the anatomical region.

12. The method of claim 9, wherein the flexible tube includes a working channel configured to deliver a second one or more tools to the anatomical region and an irrigation channel configured to irrigate the anatomical region, and
wherein a first inner diameter of the lumen is greater than each of a second inner diameter of the working channel and a third inner diameter of the irrigation channel.

13. The method of claim 9, wherein the steerable sheath includes a concentric tube structure having nested concentric tubes, and
wherein forming the bend in the steerable section is effectuated through applying an axial push-pull force to the concentric tubes.

14. The method of claim 9, wherein the steerable sheath includes a first pullwire and a second pullwire each coupled to the tip, and
wherein forming the bend in the steerable section is effectuated through applying a pull force to the first pullwire or the second pullwire.

15. A device for performing endoscopic surgery within a patient comprising:
a steerable sheath including a tip, a lumen, and a steerable section that is actuable to form a bend, such that the tip is operable to be steered toward an anatomical region within the patient;
a flexible tube including an end and a working channel that defines a fourth inner diameter of at least about two millimeters and is operable to aspirate a bodily object;
a light source disposed on the end; and
an image sensor disposed on the end,
wherein the flexible tube is disposed within the lumen such that the end is positioned at or near the tip,
wherein the steerable section includes a concentric tube structure having nested concentric tubes, and
wherein actuation of the steerable section is effectuated through applying an axial push-pull force to the concentric tubes.

16. The device of claim 15, further comprising a handle including a passage and an aspiration port in communication with the working channel, such that the working channel is operable to aspirate the bodily object from the end to the aspiration port.

17. The device of claim 15, wherein the steerable sheath has a wall thickness of between about one twentieth millimeters and one fifth millimeters.

18. The device of claim 16, wherein the steerable sheath has an outer diameter of between about two and three tenths millimeters and about four millimeters.

19. The device of claim 15, wherein the flexible tube further includes an irrigation channel configured to irrigate the anatomical region.

20. The device of claim 15, wherein the working channel is further operable to deliver a first one or more tools to the anatomical region.
